(19) United States
(12) Patent Application Publication
(10) Pub. No.: US 2005/0026849 A1
(43) Pub. Date: Feb. 3, 2005

(54) Water soluble formulations of digitalis glycosides for treating cell-proliferative and other diseases

(76) Inventors: Chandra U. Singh, San Antonio, TX (US); Robert Streeper, Marion, TX (US)

Correspondence Address: David L. Parker;Suite 2400, 600 Congress Avenue, Austin, TX, 78701, US

(21) Appl. No.: 10/812,316

(22) Filed: Mar. 29, 2004

Related U.S. Application Data
(60) Provisional application No. 60/459,466, filed on Mar. 31, 2003.

(51) Int. Cl. A61K 31/704; A61K 31/724

(52) U.S. Cl. 514/26; 514/58

(57) ABSTRACT

The present invention provides method, preparation and use of a variety of pharmaceutical composition containing at least one digitalis glycosides such as oleandrin, odoroside-A, neriifolin, proscillaridin-A, methyl-proscillaridin-A, digitoxin, digoxin and amorphous cyclodextrins. In another aspect, the present invention provides an effective method to reduce the growth of cancers or reducing the incidence of metastases. In yet another aspect, the present invention provides an effective method for treating diseases in a warm-blooded animal.

This application claims the priority of U.S. Provisional Application Ser. No. 60/459,466, filed Mar. 28, 2003, the disclosure of which application is specifically incorporated herein by reference in the entirety.

### FIELD OF THE INVENTION

The present invention is generally directed to the fields of medicine and pharmacology and is specifically related to pharmaceutical compositions, containing oleandrin and other digitalis glycosides, for use in the treatment of the cell-proliferative diseases including cancer and other diseases such as diabetes and cardiac disorder.

In another aspect, the present invention provides method, preparation and use of a variety of water soluble formulations of oleandrin and other digitalis glycosides complexed with cyclodextrins. The present invention also provides an effective method to reduce the growth of cancers or reducing the incidence of metastases.

### BACKGROUND OF THE INVENTION

Nerium Oleander is an evergreen shrub reaching four meters in height. Leaves are 10 to 22 cm long, narrow, untoothed and short-stalked, dark or grey-green in color. Some cultivars have leaves variegated with white or yellow. All leaves have a prominent mid rib, are “leathery” in texture and usually arise in groups of three from the stem. The plant produces terminal flower heads, usually pink or white, however, 400 cultivars have been bred and these display a wide variety of different flower color: deep to pale pink, lilac, carmine, purple, salmon, apricot, copper, orange and white (Huxley 1992). Each flower is about 5 cm in diameter and five-petalled. The throat of each flower is fringed with long petal-like projections. Occasionally double flowers are encountered amongst cultivars. The fruit consists of a long narrow capsule 10 to 12 cm long and 6 to 8 mm in diameter; they open to disperse fluffy seeds. Fruiting is uncommon in cultivated plants.

The plant exudes a thick white sap when a twig or branch is broken or cut (Font-Quer 1974; Schvartsman 1979; Lampe & McCann 1985; Pearn 1987). Where the species grows in the wild (i.e. in the Mediterranean), it occurs along watercourses, gravely places and damp ravines. It is widely cultivated particularly in warm temperate and subtropical regions where it grows outdoors in parks, gardens and along road sides. Elsewhere, where the plant is not frost-tolerant (e.g. in central and western Europe), it may be grown as a conservatory or patio plant. N. oleander is cultivated worldwide as an ornamental plant; it is native only in the Mediterranean region (Kingsbury 1964; Hardin & Arena 1974).

In Mediterranean region, the plant has been used extensively for medicinal purposes. For example, the macerated leaves have been used for itch and fall of hair. The fresh leaves have been applied on tumors for treatment. The decoction of leaves and bark has been used as antisyphillic. The decoction of leaves has been used as a gargle to strengthen the teeth and gum and as a nose drop for children (Dymock 1890; Chopra 1956; Dey 1984; Kirtikar 1987).

Oleander is one of the digitalis-like plants. These digitalis-like plants produce certain steroidal glycosides with cardiac properties, called as either digitalis glycosides or cardiac glycosides. Digitalis glycosides are one of the most useful groups of drugs in therapeutics (Melero 2000). For example, among the different digitalis glycosides present in Digitalis purpurea, digoxin and its derivatives (acetyl- and methyl-digoxin) are the digitalis glycosides most currently used in therapeutics.

The Oleander plant has certain toxic properties due to the presence of digitoxin like steroidal glycosides such as oleandrin. It is estimated that as many as 100 novel chemical substances are present in various parts of the Oleander plant (Krasso 1963; Siddiqui 1987-1995; Taylor 1956; Abe 1992; Hanada 1992). Oleandrin [C₃₂H₄₈O₉], a glycoside, is the main toxin in the plant. Its chemical name is 16b-acetoxy-3b-[(2,6 dideoxy-3-0-methyl-a2-L-arabino-hexopyranosyl) oxy]-14-hydroxy-5β, 14β-card-20(22)-enolide (Reynolds 1989). oleandrin forms colorless, odorless, acicular crystals which are very bitter (Shaw & Peam 1979). The concentration of oleandrin in the plant tissues is approximately 0.08% (Schvartsman 1979). oleandrin is almost insoluble in water; it has little resistance to light but it is heat-stable (Pearn 1987; Reynolds 1989). The chemical structure of oleandrin amd related digitalis glycoside is provided in Formula I. 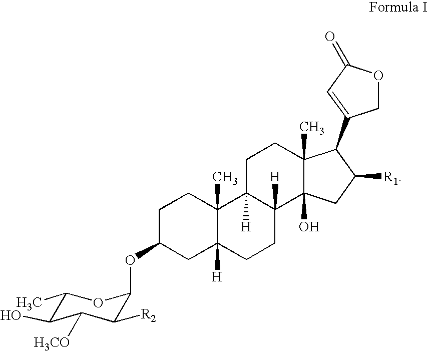
1. Oleandrin: R1=OCOCH3; R2=H
2. Neriifolin: R1=H; R2=OH
3. Odoroside A: R1=H; R2=H
4. Odoroside H: R1=H; R2=OH

Squill [Urginea maritima (L.) Baker, Liliaceae] is a native medicinal and ornamental plant from the Mediterranean area (Kopp 1996; Mitsuhashi 1994; Shoenfeld 1985; Masaru 2001). The bulbs were an ancient source of rodenticide products replaced later on by warfarin and modem anticoagulant raticides. The bulbs of these plants are enormous—often the size of one's head—and after the autumn rains they send up lush bunches of strapping great leaves. Urginea maritima is used to heal neurological pains, skin problems, deep wounds and eye afflictions. The plant also contains materials that are used in conventional medicine to treat asthma, bronchitis and heart disorders. The plant's name is derived from the root that is able to grow through hard subsoil, and reach deeply situated water. The bulb of the plant is used by the Bedouin to make poison to kill mice. It is also planted in the vicinity of Arab graves, to protect them, according to tradition. The Egyptians call the plant “Ein Sit”, the god who resists the sun, since the plant only blooms in autumn. The Bedouin believe that whenever there is an abundance of Urginea maritima flowers, there will be a rainy winter. The plant contains several cardiac glycosides including the bufadienolides proscillaridin A, scillaren A, scillirosid, gammabufotalin, and scillirosidin (Kopp 1990 & 1996; Mitsuhashi 1994; Shoenfeld 1985; Masaru 2001; Majinda 1997; Krenn 1988 & 1994; Krishna Rao 1967; Tanase 1994; Hotta 1994; Verbiscar 1986; Shimada 1979; Jha 1981; Lichti 1973).

The chemical structure of proscillaridin A and its derivative is given in formula II. In the case of proscillaridin A, a pentadienolide lactone ring is at the C17β position instead of a butenolide lactone as in oleandrin. 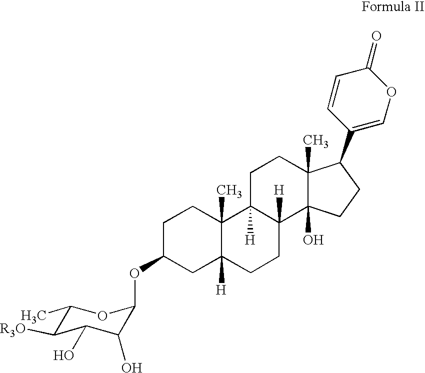
5. Proscillaridin A: R₃=H
6. Methyl-proscillaridin A: R₃=CH₃

When ingested, oleandrin gets widely distributed in the body and high concentrations of oleandrin have been measured in blood, liver, heart, lung, brain, spleen and kidney in a fatal case of N. oleander extract poisoning (Blum & Rieders 1987). Oleandrin is eliminated very slowly from the body (one to two weeks) (Shaw & Pearn 1979). In 1957, the National Cancer Institute showed that three compounds in the plant, namely, oleandrin, adynerin and ursolic acid had significant anti-cancer activities on various cancer cell lines. Since then several new chemical compounds have been identified from the methanolic or ethanolic extracts of the plant.

Proscillaridin-A is sold as a cardiatonic drug in Poland and other countries under the brand name Talusin by Knoll Pharma, Switzerland. The oral tablet form which contains 0.25 mg of proscillardin-A has a bioavailability of 20-30% in humans.

The U.S. Pat. No. 5,135,745 describes a procedure for the preparation of the extract of the plant in water. The extraction of the plant Nerium Oleander involves, cooking the leaves and stems of the plant in water for 2-3 hours and filtering off the residues. The chemical constituents of the aqueous extract have been analyzed. It has been found to contain several polysaccharides with molecular weights varying from 2KD to 30KD, oleandrin and oleandrogenin (Wang 2000). It has been shown that the water extract of the plant and oleandrin were able to induce cell killing in human cancer cells, but not in murine cancer cells and the cell-killing potency of oleandrin was greater than that of the water extract. Canine oral cancer cells treated with water extract showed intermediate levels of response, with some abnormal metaphases and cell death resulting from the treatment (Pathak 2000)

A list of cardiac glycosides from plants and toads are given in Table 1.

TABLE 1
Fanerogam and Toad species containing digitalis glycosides.

| Species | Cardiotonic glycosides |
|---|---|
| 1. Family Apocynaceae | |
| Nerium oleander | Oleandrin, neriin, neriantin. |
| Nerium odorum | Odoroside A and B. |
| Strophantus gratus, S. kombe, | Ouabain (G-strophantin), |
| S. his-pidus, | cymarin, sarmentocymarin, |
| S. sarmentosus, S. emini | periplocymarin, K-strophantin. |
| Acokanthera schimperi | Ouabain. |
| (A. ouabaïo), A. venenata, | |
| A. abyssinica | |
| Thevetia nereifolia | Thevetin, cerberin, peruvoside. |
| Thevetia yecotli | Thevetosin, thevetin A. |
| Cerbera odollam | Cerberin. |
| Cerbera tanghin | Tanghinin, |
| | deacetyltanghinin, cerberin. |
| Adenium boehmanianum | Echujin, hongheloside G. |
| 2. Family Asclepiadaceae | |
| Periploca graeca | Periplocin. |
| Periploca nigrescens | Strophantidin, strophantidol, |
| | nigrescin. |
| Xysmalobium undulatum | Uzarin. |
| Gomphocarpus fruticosus | Uzarin. |
| Calotropis procera | Calotropin. |
| 3. Family Brassicaceae | |
| Cheiranthus cheiri | Cheiroside A, cheirotoxin. |
| 4. Family Celastraceae | |
| Euonymus europaeus, E. atropur- | Eounoside, euobioside, |
| Pureus | euomonoside. |
| 5. Family Crassulaceae | |
| Kalanchoe lanceolata | Lancetoxin A and B. |
| Kalanchoe tomentosa | Kalanchoside. |
| Kalanchoe tubiforum | Bryotoxin A-C. |
| Kalanchoe pinnatum | Bryotoxin C, bryophyllin B. |
| Tylecodon wallichii | Cotiledoside. |
| Tylecodon grandiflorus | Tyledoside A-D, F and G. |
| Cotyledon orbiculata | Orbicuside A-C. |
| 6. Family Fabaceae | |
| Coronilla sp. | Alloglaucotoxin, corotoxin, |
| | coroglaucin, glaucorin. |
| 7. Family Iridaceae | |
| Homeria glauca | Scillirosidin derivatives. |
| Moraea polystachya, | Bovogenin A derivatives. |
| M. graminicola | |
| 8. Family Liliaceae | |
| Urginea scilla, U. maritima | Scillarene A and B, scilliroside, |
| | scillarenia, scilliacinoside, |
| | scilliglaucoside, scilliglaucosidin, |
| | scil-liphaeosidin, scilliphaeoside, |
| | scillirosidin, scillirubrosidin, |
| | scillirubroside, proscillaridin A. |
| Urginea rubella | Rubelin. |
| Convalaria majalis | Convalloside, convallatoxin. |
| Bowiea volubilis, B. kilimand- | Bovoside A, glucobovoside A, |
| Scharica | bovoruboside. |
| 9. Family Moraceae | |
| Antiaria africana, A. toxicaria | Antiarin a. |
| 10. Family Ranunculaceae | |
| Helleborus niger, H. viridis, | Helleborein, helleborin, hellebrin. |
| H. foeti Dus | |
| Adonis vernalis, A. aestivalis, | Adonidin, adonin, cymarin, |
| A. autumnalis, A. flammea | adonitoxin. |
| 11. Family Santalaceae | |
| Thesium lineatum | Thesiuside. |
| 12. Family Scrophulariaceae | |
| Digitalis purpurea, D. lanata | Digitoxin, gitoxin, gitalin, digoxin, |
| | F-gitonin, digitonin, lanatoside A-C. |
| 13. Toad Species Genins | |
| Bufo vulgaris | Bufotalin, bufotalinin, bufotalidin. |
| Bufo japonicus | Gamabufagin. |
| Bufo gargarizans | Cinobufagin. |
| Bufo marinus | Marinobufagin. |
| Bufo arenarum | Arenobufagin. |
| Bufo regularis | Regularobufagin. |
| Bufo valliceps | Vallicepobufagin. |
| Bufo quercicus | Quercicobufagin. |
| Bufo viridis | Viridibufagin. |
| Bufo sp. | Pseudobufotalin. |

Cardiac glycosides are used clinically to increase contractile force in patients with cardiac disorders. Their mechanism of action is well established and involves inhibition of the plasma membrane Na⁺, K⁺-ATPase, leading to alterations in intracellular K⁺ and Ca²⁺ levels.

Na⁺,K⁺-ATPase (EC 3.6.1.37) or sodium pump, is a carrier enzyme present in almost every animal cell and was discovered by Skou in 1957. Its physiological function is to maintain the Na⁺ and K⁺ electrochemical gradients through the cell membrane, keeping low Na⁺ and high K⁺ mintracellular concentrations. Such concentrations of ions, their gradients and the consequent membrane potential determine a broad range of cellular functions, as excitability of nerves and muscle cells, secondary active transport and cellular volume regulation. It is estimated to consume about 25% of total ATP consumed at rest.

Related to the transport activity, the enzyme takes out 3 Na⁺ in exchange for 2 K⁺ carried into the cell. So, it allows the restoration of the appropriate Na⁺:K⁺ ratio to maintain the transmembrane difference of potential (Na⁺ and K⁺ concentrations at rest are: [Na⁺]int.=7-20 mM, [Na⁺]ext.=140 mM, [K⁺]int.=110-120 mM, [K⁺]ext.=4-5 mM). It requires ATP and Mg²⁺ for activity. Binding of ligands to the enzyme, including a phosphorylation step, leads to conformational changes associated to Na⁺ and K⁺ transport. The supposed mechanism of action currently accepted was firstly proposed by Albers(1967) and Post(1969). This mechanism includes a step in which the enzyme, after leaving out 3 Na⁺ and before taking in 2 K⁺, can be bound, and thus inhibited, by digitalis glycosides or their analogues, preventing K⁺ binding and then stopping enzyme activity.

Na⁺,K⁺-ATPase is regulated by Na⁺ and K⁺ concentrations, as well as by several hormones, as aldosterone, thyroid hormones, catecholamines and peptide hormones (vasopresin or insulin). Hormone regulation can be carried out at different levels, from cell surface to nucleus, and it can be expressed at short or long term (Geering 1997).

Digitalis glycosides can be defined as allosteric inhibitors of Na⁺,K⁺ ATPase, and are not covalently bound to the enzyme (Repke 1989). According to the still most widely accepted mechanism of action for digitalis glycosides (Thomas 1990), they act through inhibition of Na⁺,K⁺-ATPase, thus raising indirectly the intracellular Ca²⁺ concentration ([Ca²⁺]i). Therapeutic concentrations of digitalis glycosides produce a moderate enzyme inhibition (about 30%). When the cell is depolarised, there is a lower amount of enzymes available for the restoration of the Na⁺/K⁺ balance. The remaining enzymes, non-inhibited, will act faster, because the high [Na⁺]i and the ionic balance must be restored before the following depolarisation, although it will take longer than if every enzyme were available. This lag causes a temporary increase of [Na⁺]i, reaching higher concentrations than if ATPase activity were not partially inhibited. This temporary increase of [Na⁺]i, modifies [Ca²⁺]i through a Na⁺/Ca²⁺ exchanger which allows Na⁺ exit from the cell in exchange for Ca²⁺, or Ca²⁺ exit from the cell in exchange for Na⁺, depending on the prevailing Na⁺ and Ca²⁺ electrochemical gradients (Blaustein 1974). This mechanism decreases exchange rate, or even reverses exchanger ion transport, being Ca²⁺ carried into the cell; anyway increasing [Ca²⁺]i and thus increasing contractile force.

When the concentration of digitalis glycosides reaches toxic levels, enzyme inhibition is too high (>60%), thus decreasing Na⁺ and K⁺ transport to the extent that the restoring of normal levels during diastole is not possible before the next depolarisation. Then, a sustained increase of [Na⁺]i , and thus of [Ca²⁺]i, gives rise to toxic effects (i.e. arrhythmia) of these glycosides.

Digitalis glycosides represent a very important group of drugs for the treatment of heart failure, but display a main disadvantage, which arises from their narrow therapeutic index, so they have to be administered under a strict supervision. The proximity between effective and toxic doses is the cause of severe adverse effects to appear. Na⁺,K⁺-ATPase inhibition at therapeutic doses is the cause of their positive inotropic effect, since only little changes in [Na⁺]i are required for a large effect on contractile force (Lee 1985). Apart from this activity, they can act on other physiological systems, leading to adverse effects (Gillis 1986).

Cardiac glycosides also have well known antiproliferative effects on tumor cells (Shjratori 1967; Repke 1988 & 1995). Some cardiac glycosides have been evaluated in short term animal models. The conclusion from these experiments is that very high doses, probably toxic, would be needed for obtaining anticancer effects in humans (Cassady 1980). In contrast, recently it has been found that non-toxic concentrations of digitoxin and digoxin inhibits growth and induce apoptosis in different human malignant cell lines, whereas highly proliferating normal cells were not affected (Haux 1999 & 2000). The capability of cardiac glycosides to induce apoptosis has recently been confirmed in other studies (Kawazoe 1999). There is a great difference in susceptibility for cardiac glycosides in different species indicating that one can not extrapolate the results from animal models into humans (Repke 1988). In vitro experiments the apoptosis-inducing effect was more potent for digitoxin than for digoxin, and for digitoxin there was a dose response pattern; the higher concentration the more apoptosis. Another recent report on the anticancer effects of different cardiac glycosides on tumor cell lines also confirms that digitoxin seems more potent than digoxin (Johansson 2001).

It has been shown that cardiac glycosides oleandrin, Ouabain, and Digoxin induce apoptosis in androgen-independent human prostate cancer cell lines in vitro. Cell death was associated with early release of cytochrome c from mitochondria, followed by proteolytic processing of caspases 8 and 3. oleandrin also promoted caspase activation, detected by cleavage poly (ADP-ribose) polymerase and hydrolysis of a peptide substrate (DEVD-pNA). Comparison of the rates of apoptosis in poorly metastatic PC3 M-Pro4 and highly metastatic PC3 M-LN4 subclones demonstrated that cell death was delayed in the latter because of a delay in mitochondrial cytochrome c release. Single-cell imaging of intracellular Ca²⁺ fluxes demonstrated that the proapoptotic effects of the cardiac glycosides were linked to their abilities to induce sustained Ca²⁺ increases in the cells. These results show that cardiac glycosides can be used to the treatment of metastatic prostate cancer (McConkey 2000).

The saponin digitonin, the aglycone digitoxigenin and five cardiac glycosides were evaluated for cytotoxicity using primary cultures of tumor cells from patients and a human cell line panel (representing different cytotoxic drug-resistance patterns). Of these seven compounds, proscillaridin-A was the most potent (IC(50): 6.4--76 nM), followed by digitoxin, and then ouabain, digoxin, lanatoside C, digitoxigenin and digitonin. Correlation analysis of the log IC(50) values for the cell lines in the panel showed that compound cytotoxicity was only slightly influenced by resistance mechanisms that involved P-glycoprotein, topoisomerase II, multidrug resistance-associated protein and glutathione-mediated drug resistance. Digitoxin and digoxin expressed selective toxicity against solid tumor cells from patients, while proscillaridin-A expressed no selective toxicity against either solid or hematological tumor cells. The results revealed marked differences in cytotoxicity between the cardiac glycosides, both in potency and selectivity, and modes of action for cytotoxicity that differ from that of commonly used anticancer drugs (Johansson 2001).

Further it is known that in vitro, cardiac glycosides may inhibit fibroblast growth factor-2 (FGF-2) export through membrane interaction with the Na⁺,K⁺ ATPase pump (Yeh 2001). It has been shown that oleandrin (0.1 ng/mL) produced a 45.7% inhibition of FGF-2 release from PC3 cells and a 49.9% inhibition from DU145 cells. The water extract of the oleander plant (100 ng/mL) produced a 51.9 and 30.8% inhibition of FGF-2 release, respectively, in the two cell lines. These results demonstrate that the water extract, like oleandrin, inhibited FGF-2 export in vitro, through its interaction with Na⁺,K⁺ ATPase, from PC3 and DU145 prostate cancer cells in a concentration- and time-dependent fashion and may, therefore, contribute to the antitumor activity of the treatment for cancer (Smith 2001)

U.S. Pat. No. 6,071,885 claims cardiac glycosides, specifically, digoxin and ouabain, for the treatment of FGF-mediated pathophysiological condition in a patient. The pathophysiological condition is selected from melanoma, ovarian carcinoma, teratocarcinoma and neuroblastoma. However, the patent does not address the Na⁺,K⁺,ATPase pump inhibiting properties of these glycosides which are responsible for the FGF export inhibition (Yeh 2001). For example, Stewart et al (2000) and Grimes et al (1995) discusses the importance of the pump inhibition of these glycosides in prostate cancer cell lines. U.S. Pat. No. 6,281,197 similarly claims cardiac glycosides, especially digoxin and ouabain, for the treatment of complications of diabetes involving the inhibition of the export of leaderless FGF proteins. However, a literature search on the internet using PUBMED site for cardiac glycoside and diabetes produced more than 300 publications and all of these publications imply the importance of Na⁺,K⁺-ATPase in diabetes mellitus. It has been shown that streptozotocin-induced diabetes mellitus in the rat is associated with a substantial increase in ouabain-sensitive ATPase activity along most of the nephron (Wald 1986). Further, it has been found that there is decrease in Na⁺-K⁺ pump concentration in nerve cells in diabetic rats and the decrease may be due to atrophy of the axons. In skeletal muscles, myocardium, and peripheral nerves, the observed decrease in Na⁺-K⁺ pump concentration may be important for the pathophysiology of diabetes (Kjeldsen 1987). Diabetic neuropathy is a degenerative complication of diabetes accompanied by an alteration of nerve conduction velocity (NCV) and Na⁺,K⁺-ATPase activity. Na⁺,K⁺-ATPase activity was significantly lower in sciatic nerve membranes of diabetic rats and significantly restored in diabetic animals that received fish oil supplementation. Diabetes induced a specific decrease of alpha1- and alpha3-isoform activity and protein expression in sciatic nerve membranes(Gerbi 1998). It has been observed that high glucose with suppressed Na⁺/K⁺ pump activity might induce an increase of Ca²⁺ influx through either Ca²⁺ channels or reverse Na⁺/Ca²⁺-exchange, possibly leading to the elevation of Ca²+-activated voltage-dependent K⁺ channels. Both a decrease in inward Na⁺current and an increase in K⁺ conductance may result in decreased nerve conduction. In addition, a possible increase of axoplasmic Ca²⁺ concentration may lead to axonal degeneration. These results provide a clue for understanding the pathophysiologic mechanism of diabetic neuropathy (Takigawa 2000).

Further it has been reported that there is a reduction in activity of the ouabain-sensitive Na⁺,K⁺-ATPase pump and a reduction in membrane permeability on the diabetic erythrocyte which is most marked in Type 1 diabetics (Jennings 1986). Further it has been found that the Na⁺-pumping activity, estimated from both Na⁺,K⁺-ATPase and ouabain binding, was significantly decreased in IDDM and NIDDM subjects, but its insulin sensitivity was retained only in young IDDM subjects (Baldini 1989). It has been observed that VSMC grown in high glucose concentration milieu manifests a decreased Na-K, and Ca transport in conjunction with an increase in intracellular concentration of Na and [Ca]i. These results suggest that high glucose, per se, may alter membrane permeability to cations, possibly leading to changes in VSMC contractility and/or proliferation. This abnormality seen in the diabetic state may closely link to the pathogenesis of diabetic angiopathy, thus as a result risking hypertension and vascular disease (Kuriyama 1994). Sennoune et al (2000) studied in rats the effect of streptozotocin-induced diabetes on liver Na⁺,K⁺-ATPase. Diabetes mellitus induced an increased Na⁺,K⁺-ATPase activity and an enhanced expression of the betal subunit; Diabetes mellitus led to a decrease in membrane fluidity and a change in membrane lipid composition. The results suggest that the increase of Na⁺,K⁺-ATPase activity can be associated with the enhanced expression of the betal subunit in the diabetic state, but cannot be attributed to changes in membrane fluidity as typically this enzyme will increase in response to an enhancement of membrane fluidity. Further, the level of Na⁺,K⁺-ATPase activity and the number of enzyme units were about 30% lower in the red blood cells of diabetic patients than in healthy Caucasian controls (Raccah 1996).

The adenosine triphosphate-binding site, investigated by anisotropy decay studies of the fluorescent probe pyrene isothiocyanate, was modified in women with IDDM and it appears that the Na⁺,K⁺-ATPase of human placenta is altered in its disposition in IDDM (Zolese 1997). The alterations in small intestinal Na⁺,K⁺-ATPase expression in the chronic diabetic state appear to involve alterations in transcriptional and posttranscriptional events and may likely represent an adaptive response that leads to increased Na⁺-coupled monosaccharide absorption in the context of a perceived state of nutrient depletion (Wild 1999).

U.S. Pat. No. 5,872,103 describes a method for the prevention of mammary tumors by the administration of cardiac glycoside, especially, digoxin and digitoxin. The patent is directed to a method for the prevention of neoplasms which involves using a cardiac glycoside prophylactically to treat an individual who is at risk of developing a neoplasm prior to the development of a tumor in vivo.

Further, agents that can suppress the activation of nuclear factor-κB (NF-κB) and activator protein-1 (AP-1) may be able to block tumorigenesis and inflammation. oleandrin blocked tumor necrosis factor (TNF)-induced activation of NF-κB in a concentration- and time-dependent manner. This effect was mediated through inhibition of phosphorylation and degradation of IκBα, an inhibitor of NF-κB. The water extract of Nerium Oleander also blocked TNF-induced NF-κB activation; subsequent fractionation of the extract revealed that this activity was attributable to oleandrin. The effects of oleandrin were not cell type specific, because it blocked TNF-induced NF-κB activation in a variety of cells. NF-κB-dependent reporter gene transcription activated by TNF was also suppressed by oleandrin. The TNF-induced NF-κB activation cascade involving TNF receptor 1/TNF receptor-associated death domain/TNF receptor-associated factor 2/NF-κB-inducing kinase/IκBα kinase was interrupted at the TNF receptor-associated factor 2 and NF-κB-inducing kinase sites by oleandrin, thus suppressing NF-κB reporter gene expression. oleandrin blocked NF-κB activation induced by phorbol ester and lipopolysaccharide. Oleandrin also blocked AP-1 activation induced by TNF and other agents and inhibited the TNF-induced activation of c-Jun NH2-terminal kinase. Overall, the results indicate that oleandrin inhibits activation of NF-κB and AP-1 and their associated kinases. These results may provide a molecular basis for the ability of oleandrin to suppress inflammation and perhaps tumorigenesis. (Manna 2000)

While the water extract of the Nerium Oleander plant has shown to ameliorate the cell proliferative diseases in humans, it is rather difficult to develop the extract as a parenteral pharmaceutical product suitable for commercialization due to the presence of several compounds. Since the anti-tumor activity of the oleander extract has been shown to be due to the presence of oleandrin and oleandrogenin in the extract it is desirable to develop oleandrin as an anti-tumor agent. The term cell-proliferative diseases is meant here to denote malignant as well as non-malignant cell populations which often appear morphologically to differ from the surrounding tissue.

As described before, oleandrin is extremely toxic due to its cardiac properties and it is believed that the non-toxic nature of the water extract is due to the encapsulation of the water insoluble oleandrin and oleandrogenin molecules into the polysaccharides present in the extract. The encapsulated oleandrin and oleandrogenin is soluble in water and oleandrin is released slowly upon administration through injection. Also, the amount of oleandrin encapsulated by the extraction procedure is very small (2-5 microgram per mg) and it should be possible to develop alternate delivery vehicles to reduce the toxicity of oleandrin and other digitalis glycosides and thereby increase its therapeutic value. It is highly desirable to develop new procedures for the increase of the therapeutic value of oleandrin and other digitalis glycosides to treat cancers in humans.

There are many potential barriers to the effective delivery of a toxic drug in its active form to solid tumors. Most small-molecule chemotherapeutic agents have a large volume of distribution on intravenous administration. The result of this is often a narrow therapeutic index due to a high level of toxicity in healthy tissues. Through encapsulation of drugs in a macromolecular carrier, such as a liposome, the volume of distribution is significantly reduced and the concentration of drug in the tumor is increased. This results in a decrease in the amount and types of nonspecific toxicities and an increase in the amount of drug that can be effectively delivered to the. Under optimal conditions, the drug is carried within the liposomal aqueous space while in the circulation but leaks at a sufficient rate to become bioavailable on arrival at the tumor. The liposome protects the drug from metabolism and inactivation in the plasma, and due to size limitations in the transport of large molecules or carriers across healthy endothelium, the drug accumulates to a reduced extent in healthy tissues. However, discontinuities in the endothelium of the tumor vasculature have been shown to result in an increased extravasation of large carriers and, in combination with an impaired lymphatics, an increased accumulation of liposomal drug at the tumor. All of these factors have contributed to the increased therapeutic index observed with liposomal for-mulations of some chemotherapeutic agents (Drummond et al 1999).

Protein microspheres have also been reported in the literature as carriers of pharmacological or diagnostic agents. Microspheres of albumin have been prepared by either heat denaturation or chemical crosslinking. Heat denatured microspheres are produced from an emulsified mixture (e.g., albumin, the agent to be incorporated, and a suitable oil) at temperatures between 100° C. and 150° C. The microspheres are then washed with a suitable solvent and stored. Leucuta et al.,(1988) describe the method of preparation of heat denatured microspheres. The procedure for preparing chemically crosslinked microspheres involves treating the emulsion with glutaraldehyde to crosslink the protein, followed by washing and storage. Lee et al., (1981) and U.S. Pat. No. 4,671,954 teach this method of preparation. The above techniques for the preparation of protein microspheres as carriers of pharmacologically active agents, although suitable for the delivery of water-soluble agents, are incapable of entrapping water-insoluble ones. This limitation is inherent in the technique of preparation which relies on crosslinking or heat denaturation of the protein component in the aqueous phase of a water-in-oil emulsion. Any aqueous-soluble agent dissolved in the protein-containing aqueous phase may be entrapped within the resultant crosslinked or heat-denatured protein matrix, but a poorly aqueous-soluble or oil-soluble agent cannot be incorporated into a protein matrix formed by these techniques.

U.S. Pat. Nos. 5439686 and 5916596 teach the methods for the production of particulate vehicles for the intravenous administration of pharmacologically active agents. They disclose methods for the in vivo delivery of substantially water insoluble anticancer drug taxol. The suspended particles are encased in a polymeric shell formulated from a biocompatible polymer, and have a diameter of less than about 1 micron. The polymeric shell contains particles of taxol, and optionally a biocompatible dispersing agent in which pharmacologically active agent can be either dissolved or suspended.

Another approach as has been to form a reversible complex between the insoluble drug, such as oleandrin, and a carrier molecule. The characteristics of the carrier molecule are such that the carrier molecule and the reversible complex are soluble in water. Among these known carrier molecules are cyclodextrin compounds. The use of cyclodextrin derivatives as carrier molecules for pharmaceutics is reviewed by Albers and Muller (Albers 1995).

A variety of improvements in the characteristics of pharmaceutical complexes including various cyclodextrins and cyclodextrin derivatives are disclosed in the following U.S. Pat. Nos.: Noda et al., U.S. Pat. No. 4,024,223 methyl salicylate; Szejtli et al U.S. Pat. No. 4,228,160 indomethacin; Hyashi et al., U.S. Pat. No. 4,232,009 ω-halo-PGI₂ analogs; Matsumoto et al., U.S. Pat. No. 4,351,846 3-hydroxy and 3-oxo prostaglandin analogs; Yamahira et al., U.S. Pat. No. 4,353,793, bencyclane fumarate; Lipari, U.S. Pat. No. 4,383,992 steroids-corticosteroids, androgens, anabolic steroids, estrogens, progestagens complexed with β-cyclodextrin, but not substituted amorphous β cyclodextrins; Nicolau, U.S. Pat. No. 4,407,795 P-hexadecylaminobenzoic acid sodium salt; Tuttle, U.S. Pat. No. 4,424,209 3,4-diisobutyryloxy-N-[3-(4-isobuttyryloxyphenyl)-1-methyl-n-propyl]-β-phenethylamine, Tuttle, U.S. Pat. No. 4,425,336, 3,4-dihydroxy-N-[3-(4-hydroxyphenyl)-1-methyl-n-propyl]-β-phenethylamine; Wagu et al., U.S. Pat. No. 4,438,106 fatty acids EPA and DHA; Masuda et al., U.S. Pat. No. 4,474,881 2-(2-fluoro-4-biphenyl)propionic acid or salt; Shinoda et al., U.S. Pat. No. 4,478,995 acid addition salt of (2′-benzyloxycarbonyl)phenyl trans-4-guanidinomehtylcyclo-hexanecaboxylate; Hyashi et al., U.S. Pat. No. 4,479,944 Prostaglandin I₂ analog; Hayashi et al., U.S. Pat. No. 4,479,966, 6,9-methano-prostaglandin I₂ analogs; Harada et al., U.S. Pat. No. 4,497,803 lankacidin-group antibiotic; Masuda U.S. Pat. No. 4,499,085 prostoglandin analog; Szejtli et al., U.S. Pat. No. 4,524,068 piperonyl butoxide; Jones, U.S. Pat. No. 4,555,504 cardiac glycoside; Uekama et al., U.S. Pat. No. 4,565,807 pirprofen; Ueda et al., U.S. Pat. No. 4,575,548 2-nitroxymethyl-6-chloropyridine; Ohwaki et al., U.S. Pat. No. 4,598,070 tripamide anti-hypertensive; Chiesi et al., U.S. Pat. No. 4,603,123 piroxicam (feldene); Hasegawa et al., U.S. Pat. No. 4,608,366 monobenzoxamine; Hiari et al., U.S. Pat. No. 4,659,696 polypeptide; Szejtili et al., U.S. Pat. No. 4,623,641 Prostoglandin I₂ methyl ester; Ninger et al., U.S. Pat. No. 4,663,316. unsaturated phosphorous containing antibiotics including phosphotrienin; Fukazawa et al., U.S. Pat. No. 4,675,395 hinokitol; Shimizu et al., U.S. Pat. No. 4,728,509 3-amino-7-isopropyl-5-oxo-5H-[1]-benzopyrano[2,3-b]pyridine-3-carboxcylic acid; Shibani et al. U.S. Pat. No. 4,728,510 milk component; and Karl et al., U.S. Pat. No. 4,751,095 aspartame.

Among the above-mentioned patents, several indicate that complexes of cyclodextrin with drug substances improve side effects of the drug substance. Szejtli et al., U.S. Pat. No. 4,228,160 disclosed that the frequency and severity of gastric and duodenal erosion and ulceration in rats caused by indomethecin is improved in an oral formulation of a complex of β-cyclodextrin: indomethacin in a 2:1 ratio, but is not improved and in fact worsens in the same oral formulation of a complex of β-cyclodextrin: indomethacin in a 1:1 ratio.

Shimazu et al., U.S. Pat. No. 4,352,793 discloses that a formulation wherein bencyclane fumnarate an anti-convulsive compound and β-cyclodextrin or γ-cyclodextrin yields a complex in which the bencyclane fumarate is an inclusion compound. These complexes, when formulated as a liquid suitable for oral administration were claimed to be less irritating in an isotonic buffered pH 7 solution when administered as drops to the eyes of rabbits, as compared to bencyclane fumarate drops at the same drug concentration. Shimazu et al., also discloses that similar complexes dissolved in rabbit blood in vitro yielded reduced hemolysis as compared to equal concentrations of bencyclane fumarate alone mixed with rabbit blood.

Masuda et al., U.S. Pat. No. 4,478,811 disclose ophthalmic formulations of β- or γ-cyclodextrin complexes of the nonsteroidal anti-inflammatory compound fluoro-bi-phenylacetic acid which are less irritating and painful than the same formulations of fluoro-bi-phenyl acetic acid alone.

Uekama et al., U.S. Pat. No. 4,565,807 discloses complexes of α-, β- and γ-cyclodextrin, piprofen and a pharmaceutically acceptable base. Piprofen is an analgesic and anti-inflammatory compound which is bitter and can cause irritation to the gastrointestinal tract. The complexes disclosed in the patent have improved less bitter taste and are less gastrointestinal irritating than the uncomplexed compound piprofen. No preparations suitable for intravenous injection were disclosed.

Lipari, U.S. Pat. No. 4,383,992 discloses topical and ophthalmic solutions comprising a number of different steroid-related compounds including corticosteroids, androgens, anabolic steroids, estrogens, and progestagens complexed with β cyclodextrin. None of the cyclodextrin compounds disclosed by Lipari are substituted or amorphous cyclodextrins. In addition, none or the steroid related compounds disclosed by Lipari are 5β steroids.

Pitha et al., U.S. Pat. No. 4,596,795 discloses complexes containing amorphous hydroxypropyl-β-cyclodextrin and sex hormones, particularly testosterone, progesterone and estradiol as a lyophilized powder in tablet form. These tableted complexes are disclosed as appropriate for administration sublingually or bacilli with absorption occurring across the corresponding mucosal membrane. None is administered in solution parenterally. In addition none of the steroid related compounds disclosed by Pitha et al are 5β steroids.

Pitha et al., U.S. Pat. No. 4,727,064 discloses complexes containing water soluble amorphous substituted cyclodextrin mixtures and drugs with substantially low water solubility which may be lyophilized and the lyophilized powder formed into dosage forms suitable for absorption trans-mucocele across the oral, buccal or rectal mucosa. The solutions of amorphous water soluble cyclodextrin alone and not in a complex with a drug substance are disclosed as nonirritating topically, and having low toxicity, both systemic and local, when applied parenterally. These solutions of substituted cyclodextrin alone were tested and shown to be non-lethal when substantial amounts of the cyclodextrin solution were administered intra peritoneally in mice. A number of categories of drugs are disclosed in Pitha et al. U.S. Pat. No. 4,727,064 for complex with cyclodextrin derivatives and include inter alia vitamins, salts of retinoid acid, spironolactone, antiviral agents, diuretics, anticoagulants, anticonvulsant and anti-inflammatory agents. Significantly, Pitha et al. U.S. Pat. No. 4,727,064 while disclosing that aqueous solutions of 50% cyclodextrin may be used for the purpose of determining solubility of drugs in such solutions does not indicate that such solutions are suitable for intravenous administration. No attempt is made to qualify the solution as to its pyrogenicity. The claimed compositions of matter in the reference contain only cyclodextrin and drug. Liquid or semi-liquid compositions of matter, which are claimed in the reference, appear to be made of cyclodextrins with higher degrees of substitution with hydroxy propyl groups. These cyclodextrins are themselves semi-solid or liquids according to the reference. Thus no aqueous formulations of water, cyclodextrin and drug are disclosed or claimed as suitable for parenteral administration in the reference.

Bekers et al. (1989) describes the investigation of stabilization of mitomycin-C and several related mitomycins by formation of a complex with cyclodextrin. The authors indicate that at the pH ranges studied β- and β-cyclodextrin as well as heptakis-(2,6,-di-O-methyl)-β-cyclodextrin and dimethyl-β-cyclodextrin, have no influence on stabilization of mitomycin-C pH degradation. γ-cyclodextrin is reported as having measurable stabilizing effect on mitomycin in acidic media at pH above 1.

Bodor, U.S. Pat. No. 5,024,998 and Bodor, U.S. Pat. No. 4,983,586 disclose a series of compositions comprising complexes of hydroxypropyl-β-cyclodextrin (HPCD) complexed to a difficult to solubilize drug, or HPCD complexed to a drug which has first been complexed to a specific class of drug carriers characterized as redox drug carriers. The complex of drug and redox carrier is itself difficult to solubilize and is highly lipophilic due to the presence of pyridine derivatives as part of the redox carrier complex. Bodor 5,024,998 and 4,983,586 further claim that a solution of 20 to 50% hydroxypropyl-β-cyclodextrin and lipophilic drug-redox carrier complex, or 20 to 50% hydroxypropyl-β-cyclodextrin and lipophilic and/or water labile drug is useful in a method of “decreasing the incidence of precipitation of a lipophilic and/or water labile drug occurring at or near the injection site and/or in the lungs or other organs following parenteral administration.” Significantly the Bodor references attribute the precipitation and organ deposition problems associated with parenteral administration of lipophilic drugs to the effects of organic solvents used to solubilized the drug in the parenteral vehicle. The Bodor references additionally state that drugs which are particularly useful in the parenteral composition and methods disclosed therein are those which are relatively insoluble in water but whose water solubility can be substantially improved by formulation with 20 to 50% of the selected cyclodextrin, e.g., HPCD, in water. Significantly no part of Bodor addresses the pyrogenic load on the cyclodextrin or the issue of the pyrogenic effect of the composition when injected parenterally. Thus it is quite clear that the Bodor references are directed to prevention of the phenomenon of precipitation of insoluble drugs and insoluble drug-carrier complexes.

U.S. Pat. No. 5,824,668 discloses the composition of 5β steroid with cyclodextrin suitable for parenteral administration for treating various diseases.

Muller et al (1992) describes the complex formation of digitoxin with β- and γ-cyclodextrins. Uekama et al (1983) describes the inclusion complexes of the digitalis glycosides digitoxin, digoxin, and methyl digoxin with three cyclodextrins (α-, β-, γ-homologues) in water and in the solid state were studied by a solubility method, IR and 1H-NMR spectroscopy, and X-ray diffractometry. Solid complexes (in a molar ratio of 1:4) of the digitalis glycosides with γ-cyclodextrin were prepared and their in vivo absorption examined. The rapidly dissolving form of the γ-cyclodextrin complex significantly increased plasma levels of digoxin (approximately 5.4-fold) after oral administration to dogs. Ueda et al (1999) examined the complex formation of digitoxin with delta-cyclodextrin and observed enhanced solubility. Okada and Koizumi (1998) studied the complex formation of digitoxin and digoxin with modified β-cyclodextrins. None of the above studies address the issue of parenteral administration of the digitalis glycosides complexed with cyclodextrins.

Further there are no scientific studies on the complex formation of cyclodextrins with oleandrin or other digitalis glycosides such as neriifolin, odoroside and proscillaridin-A.

U.S. Pat. No. 6407079 discloses the pharmaceutical compositions comprising inclusion compounds of sparingly water-soluble or water-instable drugs with β-cyclodextrin ethers or β-cyclodextrin esters and the process for the preparation of such compositions. The patent claims cardiac glycoside as one of the drug for the treatment of cardiac disorder and the molar ratio of the drug to the cyclodextrin derivative is from about 1:6 to 4:1. The patent claims injectable formulations with 0.45 micron filtering and sterilization. However, the patent does not address the pyrogenicity of the preparation and there is no example of the preparation of the cardiac glycoside-cyclodextrin complex suitable for parenteral administration. According to the patent document, the patent was being filed in 1988 and has been awarded in 2002. However, the complexation of digitoxin and digoxin with β- and γ-cyclodextrins have been disclosed to the public by the inventors in 1992 (Muller et al 1992).

The present invention addresses the parenteral and oral administration of the water soluble formulation of the compound selected from the digitalis type of digitalis glycosides such as oleandrin, odoroside A and H, neriifolin, proscillaridin A, digitoxin, digoxin complexed with cyclodextrins.

### SUMMARY OF THE INVENTION

The present invention relates to the water soluble formulations of digitalis type of cardiac glycosides such as oleandrin, digitoxin, digoxin suitable for parenteral administration. In particular embodiment, the invention relates to the use of the digitalis glycosides as anti-tumor agents. The inventors have demonstrated that the water soluble formulations of the digitalis glycosides such as oleandrin, disclosed herein, for example, exerts cytotoxic effects in human cancer cell lines and in animals transplanted with these cancer cells.

In a preferred embodiment the composition of the present invention, comprises at least one digitalis glycoside such as oleandrin. It will, of course, be understood that the composition may further comprise a second digitalis glycoside or one or more other pharmacologically-active compounds, and particularly one or more anti-tumor compounds. The methods of the invention may thus entail the administration of one, two, three, or more, of digitalis glycosides such as oleandrin. The maximum number of species that may be administered is limited only by practical considerations, such as the particular effects of each compound.

In yet another aspect, the present invention provides an effective method for treating diseases such as inflammation, cancer, arthritis, cardiac disorder and diabetes in a warm-blooded animal.

This invention also provides a method for producing water soluble formulations of digitalis glycosides such as oleandrin which can be sterile filtered through a 0.22 μm filter.

In yet another embodiment of the method, the sterile-filtered water soluble formulations of digitalis glycosides can be lyophilized in the form of a cake in vials using cryoprotectants such as sucrose, mannitol, trehalose or the like. The lyophized cake can be reconstituted to the original formulations. These water soluble formulations are administered by a variety of routes, preferably by intravenous, parenteral, intratumoral and oral routes.

The invention also includes the method for delivering the water soluble formulations of digitalis glycosides orally by making capsules or tablets containing the lyophilized powder of the digitalis glycoside with cyclodextrins.

The invention also includes a method of treating cancer with digitalis glycosides. This method comprises administration of an effective amount of a suitable water soluble formulation containing the digitalis glycosides to a subject in need thereof. Administration is preferably by either intramuscular or intravenous injections or by oral route. The treatment may be maintained as long as necessary and may be used in conjunction with other forms of treatment.

### DETAILED DESCRIPTION OF THE INVENTION

It is understood as “digitalis activity” the ability to inhibit Na⁺,K⁺-ATPase through acting onto the digitalis receptor, along with the ability to display a positive inotropic effect. Such an action is performed by several natural, semisynthetic and synthetic compounds (Thomas 1992). Among the natural compounds, there are three groups: steroidal butenolides and pentadienolides, known as “cardiotonic steroids” or “digitalic compounds” and Erythrophleum alkaloids. The word “digitalis” is often used as a generic word for all cardiotonic steroids; similarly, the receptor for these compounds is generally known as “digitalis receptor”. Digitalis glycosides or also called as cardiac glycosides are compounds bearing a steroidal genin or aglycone with one or several sugar molecules attached to position C-3. In the case of toad venom, sugar is replaced by suberylarginine.

As used herein, the term “micron” refers to a unit of measure of one one-thousandth of a millimeter.

As used herein, the term “nm” or the term “nanometer” refers to a unit of measure of one one-billionth of a meter.

As used herein, the term “ng” or the term “nanogram” refers to a unit of measure of one one-billionth of a gram.

As used herein, the term “mL” refers to a unit of measure of one one-thousandth of a liter.

As used herein, the term “mM” refers to a unit of measure of one one-thousandth of a mole.

As used herein, the term “biocompatible” describes a substance that does not appreciably alter or affect in any adverse way, the biological system into which it is introduced.

As used herein, the term “substantially water insoluble pharmaceutical agent” means biologically active chemical compounds which are poorly soluble or almost insoluble in water. Examples of such compounds are paclitaxel, oleandrin, cyclosporine, digitoxin and the like.

By cyclodextrin is meant α-, β-, or γ-cyclodextrin. Cyclodextrins are described in detail in Pitha et al., U.S. Pat. No. 4,727,064 which is incorporated herein by reference. Cyclodextrins are cyclic oligomers of glucose; these compounds form inclusion complexes with any drug whose molecule can fit into the lipophile-seeking cavities of the cyclodextrin molecule.

The term cell-proliferative diseases is meant here to denote malignant as well as non-malignant cell populations which often appear morphologically to differ from the surrounding tissue.

By amorphous cyclodextrin is meant non-crystalline mixtures of cyclodextrins wherein the mixture is prepared from α-, β-, or γ-cyclodextrin. In general the amorphous cyclodextrin is prepared by non-selective additions, especially alkylation of the desired cyclodextrin species. Reactions are carried out to yield mixtures containing a plurality of components thereby preventing crystallization of the cyclodextrin. Various alkylated and hydroxyalkyl-cyclodextrins can be made and of course will vary, depending upon the starting species of cyclodextrin and the addition agent used. Among the amorphous cyclodextrins suitable for compositions according to the invention are hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin. In the compositions according to the invention hydroxy-β-cyclodextrin is preferred. The substituted γ-cyclodextrins may also be suitable, including hydroxypropyl, hydroxyethyl, glucosyl, maltosyl and maltotriosyl derivatives of γ-cyclodextrin.

The cyclodextrin of the compositions according to the invention may be α-, β-, or γ-cyclodextrin. α-cyclodextrin contains six glucopyranose units; β-cyclodextrin contains seven glucopyranose units; and γ-cyclodextrin contains eight glucopyranose units. The molecule is believed to form a truncated cone having a core opening of 4.7-5.3 Å, 6.0-6.5 Å and 7.5-8.3 Å in α-, β-, or γ-cyclodextrin respectively. The composition according to the invention may comprise a mixture of two or more of the α-, β-, or γ-cyclodextrins. Usually, however the composition according to the invention will comprise only one of the α-, β-, or γ-cyclodextrins. The particular α-, β-, or γ-cyclodextrin to be used with the particular digitalis type of cardiac glycosides such as oleandrin, digitoxin, digoxin to form the compositions according to the invention may be selected based on the known size of the molecule of the digitalis type of cardiac glycosides such as oleandrin, digitoxin, digoxin and the relative size of the cavity of the cyclodextrin compound. Generally if the molecule of the digitalis type of cardiac glycosides such as oleandrin, digitoxin, digoxin is relatively large, a cyclodextrin having a larger cavity is used to make the composition according to the invention. Furthermore, if the molecule selected from the digitalis type of cardiac glycosides such as oleandrin, digitoxin, digoxin is administered with an excipient it may be desirable to use a cyclodextrin compound having a larger cavity in the composition according to the invention.

The unmodified α-, β-, or γ-cyclodextrins are less preferred in the compositions according to the invention because the unmodified forms tend to crystallize and are relatively less soluble in aqueous solutions. More preferred for the compositions according to the invention are the α-, β-, and γ-cyclodextrins that are chemically modified or substituted. Chemical substitution at the 2,3 and 6 hydroxyl groups of the glucopyranose units of the cyclodextrin rings yields increases in solubility of the cyclodextrin compound.

Most preferred cyclodextrins in the compositions according to the invention are amorphous cyclodextrin compounds. By amorphous cyclodextrin is meant non-crystalline mixtures of cyclodextrins wherein the mixture is prepared from α-, β-, or γ-cyclodextrin. In general, the amorphous cyclodextrin is prepared by non-selective alkylation of the desired cyclodextrin species. Suitable alkylation agents for this purpose include but are not limited to propylene oxide, glycidol, iodoacetarnide, chloroacetate, and 2-diethylaminoethlychloride. Reactions are carried out to yield mixtures containing a plurality of components thereby preventing crystallization of the cyclodextrin. Various alkylated cyclodextrins can be made and of course will vary, depending upon the starting species of cyclodextrin and the alkylating agent used. Among the amorphous cyclodextrins suitable for compositions according to the invention are hydroxypropyl, hydroxyethyl, glucosyl, maltosyl and maltotriosyl derivatives of β-cyclodextrin, carboxyanidomethyl-β-cyclodextrin, carboxymethyl-β-cyclodextrin, hydroxypropyl-β-cyclodextrin and diethylamino-,β-cyclodextrin. In the compositions according to the invention hydroxypropyl-β-cyclodextrin is preferred although the α- or γ-analogs may also be suitable. The particular alkylated α-, β-, or γ-cyclodextrin to be used with the particular compound of digitalis glycosides such as oleandrin, digitoxin, digoxin and proscillaridin-A to form the compositions according to the invention will be selected based on the size of the molecule of the compound and the relative size of the cavity of the cyclodextrin compound. As with the unsubstituted cyclodextrins mentioned above, it may be advantageous to use alkylated cyclodextrin having a larger cavity when the composition according to the invention also includes an excipient. The use of a particular α-, β-, or γ-cyclodextrin with a particular digitalis type of cardiac glycosides such as oleandrin, digitoxin, digoxin and proscillaridin-A compound or the compound selected from the digitalis type of cardiac glycosides such as oleandrin, digitoxin, digoxin and proscillaridin-A and excipient in the compositions according to the invention may of course be optimized based on the effectiveness in of maintaining the compound of the digitalis type of cardiac glycosides such as oleandrin, digitoxin, digoxin and proscillaridin-A or mixture there of in solution.

As mentioned above, the compositions of matter of the invention comprise an aqueous preparation of preferably substituted amorphous cyclodextrin and one or more digitalis glycosides. The relative amounts of digitalis glycosides and cyclodextrin will vary depending upon the relative amount of each of the digitalis glycosides and the effect of the cyclodextrin on the compound. In general, the ratio of the weight of compound of the digitalis glycosides to the weight of cyclodextrin compound will be in a range between 1:1 and 1:100. A weight to weight ratio in a range of 1:5 to 1:50 and more preferably in a range of 1:10 to 1:20 of the compound selected from digitalis glycosides to cyclodextrin are believed to be the most effective for increased circulating availability of the digitalis glycoside. For example, oleandrin or proscillaridin-A in a ratio of between 1:10 and 1:50 (drug: amorphous cyclodextrin, wt:wt), and a final concentration of the injection solution of 0.3 mg/mL of oleandrin is expected to significantly reduce the toxicity as compared to free oleandrin or proscillaridin-A due to the complexation with amorphous cyclodextrin.

Importantly, if the aqueous solution comprising the digitalis glycosides and amorphous cyclodextrin is to be administered parenterally, especially via the intravenous route, the amorphous cyclodextrin will be substantially free of pyrogenic contaminants. Amorphous hydroxypropyl-β-cyclodextrin may be purchased from a number of vendors including Sigma-Aldrich, Inc. (St. Louis, Mo., USA). In addition, other forms of amorphous cyclodextrin having different degrees of substitution or glucose residue number are available commercially. A method for the production of hydroxypropyl-β-cyclodextrin is disclosed in Pitha et al., U.S. Pat. No. 4,727,064 which is incorporated herein by reference.

To produce the formulations according to the invention, a pre-weighed amount of hydroxypropyl-β-cyclodextrin compound, which is substantially pyrogen free is placed in a suitable depyrogenated sterile container. Methods for depyrogenation of containers and closure components are well known to those skilled in the art and are fully described in the United States Pharmacopeia 23 (United States Pharmacopeial Convention, Rockville, Md. USA). Generally, depyrogenation is accomplished by exposing the objects to be depyrogenated to temperatures above 400° C. for a period of time sufficient to fully incinerate any organic matter. As measured in U.S.P. Bacterial Endotoxin Units, the formulation will contain no more than 10 Bacterial Endotoxin Units per gram of amorphous cyclodextrin. By substantially pyrogen free is meant that the hydroxypropyl-β-cyclodextrin contains less than 10 U.S.P. bacterial endotoxin units per gram using the U.S.P. method. Preferably, the hydroxypropyl-β-cyclodextrin will contain between 0.1 and 5 U.S.P. bacterial endotoxin units per mg, under conditions specified in the United States Pharmacopeia 23.

Sufficient sterile water for injection is added to the substantially pyrogen free amorphous cyclodextrin until the desired concentration of hydroxypropyl-β-cyclodextrin is in solution. To this solution a pre-weighed amount of the compound selected from the digitalis type of cardiac glycosides such as oleandrin, digitoxin, digoxin is added with agitation and with additional standing if necessary until it dissolves.

The solution is then filtered through a sterile 0.22 micron filter into a sterile holding vessel and is subsequently filled in sterile depyrogenated vials and is capped. For products that will be stored for long periods of time, a pharmaceutically acceptable preservative may be added to the solution of oleandrin and hydroxypropyl-β-cyclodextrin prior to filtration, filling and capping or alternatively, may be added sterilely after filtration.

As discussed above, the present invention provides improved water soluble formulations of digitalis glycosides and methods of preparing and employing such formulations. The advantages of these water soluble formulations are that a drug is entrapped in cyclodextrin in dissolved form. These compositions have been observed to provide a very low toxicity form of the pharmacologically active agent that can be delivered in the form by slow infusions or by bolus injection or by other parenteral or oral delivery routes.

For increasing the long-term storage stability, these water soluble formulations may be frozen and lyophilized in the presence of one or more protective agents such as sucrose, mannitol, trehalose or the like. Upon rehydration of the lyophilized formulations, the solution retains essentially all the drug previously loaded. The rehydration is accomplished by simply adding purified or sterile water or 0.9% sodium chloride injection or 5% dextrose solution followed by gentle swirling of the suspension. The potency of the drug in water soluble formulation is not lost after lyophilization and reconstitution.

The digitalis glycosides in the cyclodextrin complex may be in the form of pharmaceutically acceptable salts, esters, amides or prodrugs or combinations thereof. However, conversion of inactive ester, amide or prodrug forms to an active form must occur prior to or upon reaching the target tissue or cell. Salts, esters, amides and prodrugs of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts are prepared from the free base (typically wherein the neutral form of the drug has a neutral—NH2 group) using conventional means, involving reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties which may be present on a drug are prepared in a similar manner using a pharmaceutically acceptable base such as sodium hydroxide, potassium hydroxide, ammonium hydroxide, calcium hydroxide, trimethylamine, or the like. Preparation of esters involves functionalization of hydroxyl and/or carboxyl groups which may be present within the molecular structure of the drug. The esters are typically acyl-substituted derivatives of free alcohol groups, i.e., moieties which are derived from carboxylic acids of the formula RCOOH where R is alkyl, and preferably is lower alkyl. Esters can be reconverted to the free acids, if desired, by using conventional hydrogenolysis or hydrolysis procedures. Preparation of amides and prodrugs can be carried out in an analogous manner. Other derivatives and analogs of the active agents may be prepared using standard techniques known to those skilled in the art of synthetic organic chemistry, or may be deduced by reference to the pertinent literature. In addition, chiral active agents may be in enantiomerically pure form, or they may be administered as an enantiomeric mixture.

In the present invention, the efficacy of water soluble formulations of oleandrin and proscillaridin-A of the present invention have been investigated on various systems such as human cell lines for cell proliferative activities and found to be active against tumors.

It is known that certain anionic polysaccharides (Baba, 1988), such as dextran sulphate, pustulan sulphate stimulate cell-mediated T-cell dependent immune responses without stimulating anti-body mediated immune responses that are B-cell dependent. On the other hand, unmodified polysaccharides stimulate only B-cells and certain other polysaccharides are known to stimulate both T-cell and B-cell responses under certain conditions. The polysaccharides present in water extract of the plant Nerium Oleander has been shown to contain galacturonic acids similar to pectin. These polysaccharides are claimed to be immune stimulants. Thus the formulations of the present inventions can contain suitable polysaccharides such as pectin, preferably, modified citrus pectin to provide the stimulant effect.

Further, it has been previously shown (GLYCAN STIMUATION OF MACROPHAGES IN VITRO, R. Seljelid, G. Bogwald and A. Lundwall, Experimental Cell Research 131 (1981) 121), that certain glucans, particularly such glucans containing 1,3-bound β-D-glucose entities, activate macrophages in vitro making same cytotoxic. Thus the formulations of the present inventions can contain suitable 1,3-β-D glucans and their derivatives such as phosphorylated 1,3-β-D glucan, aminated 1,3-β-D glucan, sulfated 1,3-β-D glucan and carboxymethylated 1,3-β-D glucan to provide the desired immune stimulant effect.

Previously, the effect of citrus pectin (CP), a complex polysaccharide rich in galactosyl residues, and its pH-modified derivative, modified citrus pectin (MCP) on the experimental metastasis of B16 melanoma and prostate was analyzed as described in the articles (Platt 1992; Inohara 1994; Pienta 1995 and Raloff 1995). U.S. Pat. No. 5,834,442 and U.S. Pat. No. 5,895,784 claims the oral administration of modified citrus pectin to treat prostate and melanoma cancer. It was found that co-injection of MCP with the B16-F1 cells intravenously resulted in a marked inhibition of their ability to colonize the lungs of the injected mice. The pH modification of CP results in the generation of smaller sized non-branched carbohydrate chains of similar sugar composition of the unmodified CP. MCP appears to be non-toxic, in vitro and in vivo and is sold as nutritional supplement by herbalists and natural medicine vendors.

Compositions employing the water soluble formulations of digitalis glycosides such as proscillaridin-A, digitoxin and oleandrin, will contain a biologically effective amount of digitalis glycosides. As used herein a biologically effective amount of a compound or composition refers to an amount effective to alter, modulate or reduce tumor growth or related conditions. For intravenous administration, a satisfactory result may be obtained employing the compounds in an amount within the range of from about 0.1 microgram/kg to about 100 microgram/kg, preferably from about 0.2 microgram/kg to about 50 microgram/kg and more preferably from about 0.2 microgram/kg to about 10 microgram/kg alone or in combination with one or more additional anti-tumor compounds in an amount within the range from about 0.01 mg/kg to about 50 mg/kg, preferably from about 0.05 mg/kg to about 20 mg/kg and more preferably from about 0.1 mg/kg to about 10 mg/kg both being employed together in the same intravenous dosage form or in separate oral or intramuscular or intravenous dosage forms taken at the same time. The amount of active compounds in such therapeutically useful compositions is such that a suitable dosage will be obtained.

The composition of matter according to the invention may be supplied as a dry powder or as a solution. If the composition of matter is to be injected into a subject it will be rendered sterile prior to injection. Accordingly, the composition of matter according to the invention may be supplied as a sterile cake, plug or powder or as a sterile lyophilized preparation in a sterile vial suitable for the addition of a sterile diluent, or as a sterile liquid solution in a sterile container.

The compositions of matter according to the invention may be supplied as a powder comprising the active pharmaceutical digitalis glycoside and amorphous cyclodextrin compound. If the composition is to be administered parenterally, for example intravenous, the composition of matter will be rendered sterile prior to such administration. Any of the several known means for rendering such pharmaceutical preparations sterile may be used so long as the active pharmaceutical compound is not inactivated and the complex with the amorphous cyclodextrin is not degraded. If the active pharmaceutical compound is heat stable, the composition of matter according to the invention may be heat sterilized. If the digitalis glycoside is not heat-stable but is not photo degraded the composition may be sterilized by exposure to ultraviolet light or by ionizing radiation. Alternatively, the composition of matter if in a powder form may be gas sterilized using for example ethylene oxide gas. In another alternative, the composition of matter according to the invention may be filter-sterilized using a 0.22 micron filter. If the composition of matter is an aqueous liquid, it may be filled in a sterile container and supplied as a sterile liquid ready for further dilution or injection neat. Alternatively such sterile liquids may be freeze-dried or lyophilized in a sterile container and capped.

In general the compositions of matter according to the invention will be made by dissolving the cyclodextrin in water and adding digitalis glycoside compound to the aqueous cyclodextrin solution. Excipients, if any are desired, may be added with or subsequent to adding the oleandrin or other digitalis glycoside compound. The resulting solution may be sterilized using any of the known methods appropriate to preserving the compound without significant degradation.

Preferably the solution will be sterile filtered, although other means such as terminal heat sterilization or irradiation may be employed as is known in the art, provided that the cyclodextrin compound is not significantly degraded. Alternatively, the components may be sterilized by any of the known methods appropriate to preserving the compound prior to mixing in water and may be mixed using sterile equipment and technique. The solution may be lyophilized in sterile containers and capped. Prior to use the lyophilized composition of matter may be reconstituted using sterile water for injection.

The container closure system used for containing the formulation according to the invention will also be treated to remove or destroy pyrogenic substances by means known in the art prior to filling and further processing. Thus the preferred compositions of matter according to the invention for parenteral administration, especially by the intravenous route will be nonpyrogenic. Nonpyrogenic preparations according to the invention, when administered to a subject, does not cause a febrile (basal body temperature raising) reaction. Although some bacterial endotoxin may be present, the amount is insufficient to elicit a febrile reaction. In general, such non-pyrogenic compositions will contain less than 10 U.S.P. bacterial endotoxin units per gram of product.

The formulation according to the invention may be supplied as a dry lyophilized powder as mentioned above or as a sterile non pyrogenic aqueous solution in a sterile container closure system such as a stoppered vial suitable for puncturing with a sterile syringe and needle.

Alternatively the formulation according to the invention may be supplied as a sterile non-pyrogenic aqueous solution in a sterile syringe or syringe and needle. As a sterile solution or powder it may also include a pharmaceutically acceptable preservative. The formulation according to the invention may also be included in other dosage forms in addition to those appropriate for parenteral administration. Preferably, such other dosage forms will include one or more of the digitalis glycosides. Such dosage forms may be in the form of aqueous suspensions, elixirs, or syrups suitable for oral administration, or compounded as a cream or ointment in a pharmaceutically acceptable topical base allowing the digitalis glycoside compounds to be absorbed across the skin. In addition the formulation according to the invention may be compounded in a lozenge or suppository suitable for trans-mucosal absorption.

For the intended oral mode of administration, the pharmaceutical compositions containing cyclodextrin-digitalis glycoside complex may be in the form of solid, semi-solid or liquid dosage forms, such as, for example, tablets, suppositories, pills, capsules, powders, liquids, suspensions, or the like, preferably in unit dosage form suitable for single administration of a precise dosage. The cyclodextrin-digitalis glycoside complex can be lyophilized and the lyophilized powder can be used for preparing solid dosage forms. The compositions will include an effective amount of the selected cyclodextrin-digitalis glycoside complex in combination with a pharmaceutically acceptable carrier and, in addition, may include other pharmaceutical agents, adjuvants, diluents, buffers, etc. The compounds may thus be administered orally, in dosage formulations containing conventional non-toxic pharmaceutically acceptable carriers, adjuvants and vehicles. The equivalent amount of active digitalis glycoside compound administered as cyclodextrin-digitalis glycoside complex will, of course, be dependent on the subject being treated, the subject's weight, the manner of administration and the judgment of the prescribing physician.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan mono-laurate, triethanolamine sodium acetate, triethanolamine oleate, etc. Actual methods of preparing such dosage forms are known, or will be apparent, to those skilled in this art; for example, see Remington's Pharmaceutical Sciences, referenced above. For oral administration, the composition will generally take the form of a tablet or capsule, or may be an aqueous or nonaqueous solution, suspension or syrup. Tablets and capsules are preferred oral administration forms. Tablets and capsules for oral use will generally include one or more commonly used carriers such as lactose and corn starch. Lubricating agents, such as magnesium stearate, are also typically added. When liquid suspensions are used, the active agent may be combined with emulsifying and suspending agents. If desired, flavoring, coloring and/or sweetening agents may be added as well. Other optional components for incorporation into an oral formulation herein include, but are not limited to, preservatives, suspending agents, thickening agents, and the like.

Oral dosage units preferably contain equivalent of digitalis glycoside such as oleandrin, in the cyclodextrin-digitalis glycoside complex, in the range of about 50 to not more than 1000 micrograms (μg), preferably in the range of about 100 and about 400 μg so long as the dose received by the patient is accompanied by minimal or substantially no undesirable side effects. A particularly preferred oral dosage unit contains about 250 μg equivalent oleandrin, more preferably about 150 μg equivalent oleandrin.

The pharmaceutical formulations of digitalis glycoside according to the present invention offer several advantages over the existing formulation of Oleander Extract administered parenterally. They can be intravenously administered and relatively high concentrations of oleandrin or other digitalis glycoside can be loaded into patients. Thus the frequency of dosage can be reduced. Thus within the spirit, the invention is related to improved formulations and methods of using the same when administering such formulations to patients. As mentioned herein above a number of excipients may be appropriate for use in the formulation which comprise the composition according to the present invention. The inclusion of excipients and the optimization of their concentration for their characteristics such as for example ease of handling or carrier agents will be understood by those ordinarily skilled in the art not to depart from the spirit of the invention as described herein and claimed herein below.

The invention will now be further described with reference to the following examples. These examples are intended to be merely illustrative of the invention and are not intended to be limiting. These examples are not intended, however, to limit or restrict the scope of the present invention in any way and should not be construed as providing conditions, parameters, reagents, or starting materials which must be utilized exclusively in order to practice the art of the present invention.

### EXAMPLE 1

### Preparation of Oleandrin-Cyclodextrin Formulation

10 milligrams (mg) of oleandrin was stirred and shaken with 10 ml of water in a test tube. Appreciable quantities of compound remained out of solution after 20 minutes accumulating as white crystals at the bottom of the test tube.

100 milligrams of oleandrin was weighed and placed in a 5 mL scintillation tube. 1.5 mL of absolute ethanol was added to the tube and shaken until the oleandrin was completely dissolved. 5 grams of pyrogen free hydroxypropyl-β-cyclodextrin (sold by, Sigma-Aldrich, Inc., St. Louis, Mo., USA) was weighed on an analytical scale and placed in a graduated cylinder. Water was added with shaking until the volume reached 90 ml. The above ethanolic solution of oleandrin was added to the aqueous solution containing hydroxypropyl-β-cyclodextrin with stirring. A clear solution was obtained. Water was added to the clear solution to make the total volume to 100 mL. Thus, 1 mg oleandrin was effectively solubilized in 1 ml of 5% solution of hydroxypropyl-β-cyclodextrin. The solution was sterile-filtered through a 0.22 μm filter. The suspension was frozen below −40° C. and lyophilized. The lyophilized cake was reconstituted with sterile water for injection prior to further use.

### EXAMPLE 2

### Preparation of Oleandrin-Cyclodextrin Formulation

The previous experiment was repeated using a 2% solution of hydroxypropyl-β-cyclodextrin prepared as in Example 1. 100 mg of oleandrin was dissolved in 100 mL of water containing 2.5 grams of hydroxypropyl-β-cyclodextrin. The solution was sterile-filtered through a 0.22 μm filter. The solution was frozen below −40° C. and lyophilized. The lyophilized cake was reconstituted with sterile water for injection prior to further use.

### EXAMPLE 3

### Preparation of Odoroside-A-Cyclodextrin Formulation

The experiment in example 1 was repeated using a 100 mg of Odoroside-A instead of oleandrin. 100 mg of Odoroside-A was dissolved in 5 grams of hydroxypropyl-β-cyclodextrin in 100 mL of water. The solution was sterile-filtered through a 0.22 μm filter. The solution was frozen below −40° C. and lyophilized. The lyophilized cake was reconstituted with sterile water for injection prior to further use.

### EXAMPLE 4

### Preparation of Oleandrin-γ-Cyclodextrin Formulation

100 milligrams of oleandrin was weighed and placed in a 5mL scintillation tube. 1-2 mL of absolute ethanol was added to the tube and shaken until the oleandrin was completely dissolved. 2.5 grams of pyrogen free hydroxypropyl-γ-cyclodextrin (sold by, Sigma-Aldrich, Inc., St. Louis, Mo., USA) was weighed on an analytical scale and placed in a graduated cylinder. Water was added with shaking until the volume reached 90 ml. The above ethanolic solution of oleandrin was added to the aqueous solution containing hydroxypropyl-γ-cyclodextrin with stirring. A clear solution was obtained. Water was added to the clear solution to make the total volume to 100 mL. Thus, 1 mg oleandrin was effectively solubilized in 1 ml of 2.5% solution of hydroxypropyl-γ-cyclodextrin. The solution was sterile-filtered through a 0.22 μm filter. The suspension was frozen below −40° C. and lyophilized. The lyophilized cake was reconstituted with sterile water for injection prior to further use.

### EXAMPLE 5

### Preparation of Proscillaridin-A-Cyclodextrin Formulation

100 milligrams of Proscillaridin-A was weighed and placed in a 5 mL scintillation tube. 1-2 mL of absolute ethanol was added to the tube and shaken until the Proscillaridin-A was completely dissolved. 2 grams of pyrogen free hydroxypropyl-β-cyclodextrin (sold by, Sigma-Aldrich, Inc., St. Louis, Mo., USA) was weighed on an analytical scale and placed in a graduated cylinder. Water was added with shaking until the volume reached 90 ml. The above ethanolic solution of Proscillaridin-A was added to the aqueous solution containing hydroxypropyl-β-cyclodextrin with stirring. A clear solution was obtained. Water was added to the clear solution to make the total volume to 100 mL. Thus, 1 mg Proscillaridin-A was effectively solubilized in 1 ml of 2% solution of hydroxypropyl-β-cyclodextrin. The solution was sterile-filtered through a 0.22 μm filter. The suspension was frozen below −40° C. and lyophilized. The lyophilized cake was reconstituted with sterile water for injection prior to further use.

### EXAMPLE 6

### Preparation of Proscillaridin-A-Cyclodextrin Formulation

The previous experiment was repeated using a 5% solution of hydroxypropyl-β-cyclodextrin prepared as in Example 5. 100 mg of Proscillaridin-A was dissolved in 100 mL of water containing 5 grams of hydroxypropyl-β-cyclodextrin. The solution was sterile-filtered through a 0.22 μm filter. The solution was frozen below −40° C. and lyophilized. The lyophilized cake was reconstituted with sterile water for injection prior to further use.

### EXAMPLE 7

### Preparation of Proscillaridin-A-γ-Cyclodextrin Formulation

100 milligrams of Proscillaridin-A was weighed and placed in a 5 mL scintillation tube. 1.5 mL of absolute ethanol was added to the tube and shaken until the Proscillaridin-A was completely dissolved. 2.5 grams of pyrogen free hydroxypropyl-γ-cyclodextrin (sold by, Sigma-Aldrich, Inc., St. Louis, Mo., USA) was weighed on an analytical scale and placed in a graduated cylinder. Water was added with shaking until the volume reached 90 ml. The above ethanolic solution of Proscillaridin-A was added to the aqueous solution containing hydroxypropyl-γ-cyclodextrin with stirring. A clear solution was obtained. Water was added to the clear solution to make the total volume to 100 mL. Thus, 1 mg Proscillaridin-A was effectively solubilized in 1 ml of 2.5% solution of hydroxypropyl-γ-cyclodextrin. The solution was sterile-filtered through a 0.22 μm filter. The suspension was frozen below −40° C. and lyophilized. The lyophilized cake was reconstituted with sterile water for injection prior to further use.

### EXAMPLE 8

### Preparation of 0.1% Oleandrin Formulation

A formulation with 0.1% of oleandrin, 2.5% hydroxypropyl beta-cyclodextrin, 0.5% sodium ascorbate and 0.04% ascorbic acid as antioxidants, 0.1% methylparaben sodium and 0.01% propylparaben sodium as preservatives (Table 2) was prepared under aseptic conditions following the method given below.

TABLE 2
Formulation of Oleandrin

| Ingredient | w/v (%) | Amount | |
|---|---|---|---|
| Oleandrin | 0.1 | 0.1 | g |
| Sodium Ascorbate | 0.5 | 0.5 | g |
| Ascorbic Acid | 0.04 | 0.04 | g |
| Hydroxypropyl beta-Cyclodextrin | 2.5 | 2.5 | g |
| Methylparaben Sodium | 0.1 | 0.1 | g |
| Propylparaben Sodium | 0.01 | 0.01 | g |
| Trehalose Dihydrate | 7.0 | 7.0 | g |
| Sterile Type I Water | Qs to | 100 | mL |

The ingredients sodium ascorbate, ascorbic acid, Methylparaben Sodium and Propylparaben were purchased as USP grade materials from Spectrum Chemical and Safety Products. The ingredients Hydroxypropyl beta-Cyclodextrin and Trehalose dihydrate were purchased from Sigma Chemicals Co.

A 150 mL sterile beaker was weighed and tarred, and the ingredients, except the oleandrin and Trehalose dihydrate, listed in Table 2 were weighed and transferred directly into the beaker. Type I water was added and the volume was adjusted to 100 mL. The solution was heated to 70-80° C. in a pre-heated circulating water-bath. The amount of oleandrin listed in Table 2 was weighed and dissolved in 1-2 mL of purified ethanol in a sterile test tube. The ingredients were stirred and the ethanol solution of oleandrin was slowly added over a period of 5-10 minutes to the aqueous solution while stirring. The trehalose dihydrate was added to the solution and the resulting solution was stirred for additional 10-15 minutes to form a clear solution. The pH of the solution measured using a Orion pH meter was approximately 6.50. When required, the pH of the solution was adjusted using either 1N NaOH or 1N HCl to 6.5±0.2. The hot solution was filtered using a 0.22 μm sterile cellulose acetate bottle-top filter with a glass pre-filter attached to a sterile media receiver bottle in the laminar flow hood. A diaphragm pump (Laboport) was connected to the bottle-top filter to filter-sterilize the solution into the media bottle. Immediately after filtering the solution, the bottle-top filter was removed and a bottle-top dispenser (Dispensette II, Brinkmann) was attached to fill 5 mL of the sterile liquid at a time in 10 mL sterile glass vials in the laminar flow hood. To each one of the 10 mL vials filled, a 3-leg gray butyl rubber stopper (Wheaton) was placed in such a way that the stopper openings were exposed outside the vial's mouth. These vials were arranged in two sterile stainless steel trays, and these trays were then placed onto the freeze-dryer stoppering trays pre-cooled to −40° C. After 7 hours, the sample was freeze-dried using following the following temperature cycle:
−35° C. for 6 hours;
0° C. for 72 hours;
30° C. for 24 hours.

The vials with freeze-dried Oleandrin-Cyclodextrin complex were stoppered under a vacuum level of about 250×10⁻³ Mbar in the stoppering tray. The vacuum pump was turned off, vacuum in the stoppering tray compartment was released, and the stainless steel trays with the vials were removed from the freeze-dryer. Each one of the vials was sealed with a flip-cap aluminum seal (Wheaton) employing a hand operated E-Z crimper. The pharmaceutically formulated freeze-dried Oleandrin-Cyclodextrin complex, when stored at room temperature in the vacuum sealed vials is stable at least for about 3 to 5 years.

RECONSTITUTION TESTING: The reconstitution test of the formulated Oleandrin-Cyclodextrin complex powder in the vacuum sealed vials was performed following the procedure given below:
1. Five mL of sterile water for injection was withdrawn using a sterile 10 mL syringe with a 20 G×1″ needle attached to it.
2. The flip-cap was detached from the aluminum seal in the vial, and the exposed surface of the rubber stopper was cleaned with 70% isopropanol.
3. The water for injection from the syringe was administered into the vial. Because the powder in the vial was under vacuum, as soon as the syringe needle was inserted, the vacuum automatically withdrew the water into the vial without having to syringe's plunger.
4. After adding the water for injection, the powder was reconstituted into a clear solution within a minute.

The reconstituted Oleandrin-Cyclodextrin complex solution was used for the further studies.

STABILITY TESTING: The testing was performed by storing the vials containing the reconstituted solution at 4° C. and room temperatures. The formulated Oleandrin-Cyclodextrin complex solution was stable for at least one month when stored at these temperatures. There was no visible precipitation of particles from the solution in this period.

STERIILITY TESTING: The freeze-dried formulated Oleandrin-Cyclodextrin complex powder was reconstituted with 5 mL sterile water for injection in a laminar flow hood under aseptic conditions at the Southwest Bioscience Laboratories, San Antonio and tested in accordance with the procedure recommended by US Pharmacopeia XXIII. The formulated solution was inoculated in a culture bottle (BBL Septi-Check) containing either 70 mL Casein Digest Broth with SPS and CO₂ or Thioglycollate Broth with SPS and CO₂. The Casein Digest Broth was aerated using a 0.2 μm filter for aerobic growth. The Casein Digest and Thioglycollate Broths were incubated at 25° C. and 35° C. for 7 days, respectively. These samples were examined each day for growth and retained for 14 days before discarding. For 14 days the cultures were also observed for microbial growth after incubating under the same conditions as the samples. No microbial growth was observed in the cultures with or without formulated Oleandrin-Cyclodextrin complex solution, indicating that the freeze-dried formulated Oleandrin-Cyclodextrin complex powder was sterile.

### EXAMPLE 9

### Preparation of 0.05% Oleandrin Formulation

A formulation with 0.05% of oleandrin, 1.5% hydroxypropyl cyclodextrin, 0.5% sodium ascorbate and 0.02% ascorbic acid as antioxidants, 0.1% methylparaben sodium and 0.01% propylparaben sodium as preservatives (Table 3) was prepared under aseptic conditions following the method given in EXAMPLE 8.

TABLE 3
Formulation of Oleandrin

| Ingredient | w/v (%) | Amount | |
|---|---|---|---|
| Oleandrin | 0.05 | 0.05 | g |
| Sodium Ascorbate | 0.5 | 0.5 | g |
| Ascorbic Acid | 0.02 | 0.02 | g |
| Hydroxypropyl Cyclodextrin | 1.5 | 1.5 | g |
| Methylparaben Sodium | 0.1 | 0.1 | g |
| Propylparaben Sodium | 0.01 | 0.01 | g |
| Trehalose Dihydrate | 7.0 | 7.0 | g |
| Sterile Type I Water | Qs to | 100 | mL |

### EXAMPLE 10

### Preparation of 0.1% Proscillaridin-A Formulation

A formulation with 0.1% of proscillaridin-A, 2.5% hydroxypropyl cyclodextrin, 0.5% sodium ascorbate and 0.05% ascorbic acid as antioxidants, 0.1% methylparaben sodium and 0.01% propylparaben sodium as preservatives (Table 4) was prepared under aseptic conditions following the method given in EXAMPLE 8.

TABLE 4
Formulation of Proscillaridin-A

| Ingredient | W/v (%) | Amount | |
|---|---|---|---|
| Proscillaridin-A | 0.1 | 0.1 | g |
| Sodium Ascorbate | 0.5 | 0.5 | g |
| Ascorbic Acid | 0.05 | 0.05 | g |
| Hydroxypropyl Cyclodextrin | 2.5 | 2.5 | g |
| Methylparaben Sodium | 0.1 | 0.1 | g |
| Propylparaben Sodium | 0.01 | 0.01 | g |
| Trehalose Dihydrate | 7.0 | 7.0 | g |
| Sterile Type I Water | Qs to | 100 | mL |

### EXAMPLE 11

### Preparation of 0.1% Oleandrin Formulation with Modified Citrus Pectin

A formulation with 0.1% of oleandrin, 2.5% hydroxypropyl cyclodextrin, 0.5% sodium ascorbate and 0.05% ascorbic acid as antioxidants, 0.1% methylparaben sodium and 0.01% propylparaben sodium as preservatives and 1.5% modified citrus pectin which is derived from pectin by controlled hydrolysis (Table 5) was prepared under aseptic conditions following the method given in EXAMPLE 8.

TABLE 5
Formulation of Oleandrin with Modified Citrus Pectin

| Ingredient | W/v (%) | Amount | |
|---|---|---|---|
| Oleandrin | 0.05 | 0.05 | g |
| Sodium Ascorbate | 0.5 | 0.5 | g |
| Ascorbic Acid | 0.05 | 0.05 | g |
| Hydroxypropyl Cyclodextrin | 2.5 | 2.5 | g |
| Methylparaben Sodium | 0.1 | 0.1 | g |
| Propylparaben Sodium | 0.01 | 0.01 | g |
| Modified Citrus Pectin | 1.5 | 1.5 | g |
| Trehalose Dihydrate | 7.0 | 7.0 | g |
| Sterile Type I Water | Qs to | 100 | mL |

### EXAMPLE 12

### Preparation of 0.1% Oleandrin Formulation with Freeze Dried Polysaccharide from Oleander Plant

Preparation of Nerium Oleander Polysaccharide: The branches of nerium oleander plant grown under quarantine conditions were washed thoroughly two times with tap water, one time each with DI water and sterile Type I water ( Purity Water System, San Antonio) and then cut into pieces of about one inch. The cut stems and leaves were weighed and transferred into a 50 L glass round bottom flask, which was placed onto a mantle. To approximately 7 kg of leaves and stems, 30 L of sterile Type I water was added to the flask. A ground joint with a condenser and a thermometer was then attached to the flask, and it was heated for 4-6 hours after the mixture started boiling. The boiled oleander extract was cooled then to between 60° and 70° C. The solution was transferred, employing a peristaltic pump, into a sterile Corning 0.22 μm cellulose acetate bottle-top filter with a glass-fiber pre-filter, attached to a sterile 2 L media bottle (Corning) in a laminar flow hood (LABGRAD). A diaphragm pump (Laboport) was connected to the bottle-top filter to filter-sterilize the solution into the media bottle. Immediately after filtering about 2 L of the solution, the bottle-top filter was removed from the media bottle and the bottle was closed tightly with a cap, inside the hood. Thirteen such 2 L bottles with about 26.5 L of the filter-sterilized solution were heated to about 100° C. for 1 hour by placing these bottles in a water-bath (Precision Scientific) pre-heated to 100° C. These media bottles with the hot sterile solution were cooled to room temperature, and then stored at 4° C. in a refrigerator.

The above water extract of the oleander plant was mixed with ethanol (96%) in a 1:1 ratio. This solution was allowed to set for 12 hours, the resulting gel suspension was filtered off. The gel suspension filtrate was dissolved in distilled water. This solution was again mixed with ethanol (96%) in a 1:1 ratio. After allowing the solution to set for at least 12 hours, the gel suspension was filtered off. Again the gel suspension filtrate was dissolved in distilled water, frozen and lyophilized. In this way, the freeze dried polysaccharide extract from the oleander plant was obtained.

A formulation with 0.1% of oleandrin, 2.5% hydroxypropyl cyclodextrin, 0.5% sodium ascorbate and 0.05% ascorbic acid as antioxidants, 0.1% methylparaben sodium and 0.01% propylparaben sodium as preservatives and 1.0% freeze dried polysaccharide from the oleander plant (Table 6) was prepared under aseptic conditions following the method given in EXAMPLE 8.

TABLE 6
Formulation of Oleandrin with freeze dried polysaccharide
extract from the oleander plant

| Ingredient | W/v (%) | Amount | |
|---|---|---|---|
| Oleandrin | 0.1 | 0.1 | g |
| Sodium Ascorbate | 0.5 | 0.5 | g |
| Ascorbic Acid | 0.05 | 0.05 | g |
| Hydroxypropyl Cyclodextrin | 2.5 | 2.5 | g |
| Methylparaben Sodium | 0.1 | 0.1 | g |
| Propylparaben Sodium | 0.01 | 0.01 | g |
| Freeze dried Polysaccharide from | 1.0 | 1.0 | g |
| the Oleander Plant | | | |
| Trehalose Dihydrate | 7.0 | 7.0 | g |
| Sterile Type I Water | Qs to | 100 | mL |

### EXAMPLE 13

### Preparation of 0.1% Proscillaridin-A Formulation with Modified Citrus Pectin

A formulation with 0.1% of proscillaridin-A, 2.5% hydroxypropyl cyclodextrin, 0.5% sodium ascorbate and 0.05% ascorbic acid as antioxidants, 0.1% methylparaben sodium and 0.01% propylparaben sodium as preservatives an 1.5 Modified Citrus Pectin (Table 7) was prepared under aseptic conditions following the method given in EXAMPLE 8.

TABLE 7
Formulation of Proscillaridin-A with Modified Citrus Pectin

| Ingredient | W/v (%) | Amount | |
|---|---|---|---|
| Proscillaridin-A | 0.1 | 0.1 | g |
| Sodium Ascorbate | 0.5 | 0.5 | g |
| Ascorbic Acid | 0.05 | 0.05 | g |
| Hydroxypropyl Cyclodextrin | 2.5 | 2.5 | g |
| Methylparaben Sodium | 0.1 | 0.1 | g |
| Propylparaben Sodium | 0.01 | 0.01 | g |
| Modified Citrus Pectin | 1.5 | 1.5 | g |
| Trehalose Dihydrate | 7.0 | 7.0 | g |
| Sterile Type I Water | Qs to | 100 | mL |

### EXAMPLE 14

### Oral and Suppository Formulations of Oleandrin-Cyclodextrin Complex

100 mg of oleandrin was weighed and placed in a sterile test tube. The oleandrin was dissolved in 2-3 mL of purified absolute ethanol. 50 ml of 9.8% solution of hydroxypropyl-β-cyclodextrin was prepared in a 150 mL sterile beaker and the solution was heated to 70-80 degree centigrade while stirring on a hot plate. The ethanolic solution of oleandrin was slowly added to the beaker with stirring. Within 10-30 minutes, the oleandrin dissolved, leaving a clear solution with no accumulation of crystals. Thus, 100 mg was effectively solubilized in 50 ml of 9.8% solution of hydroxypropyl-β-cyclodextrin. The solution was sterile-filtered through a 0.22 μm filter. The solution was frozen below −40° C. and lyophilized. The lyophilized cake was powdered and used for the tablets, capsule and coated pills formulations and the lyophilized powder is denoted as oleandrin-cyclodextrin complex.

A. Preparation of Tablets

The tablet composition is compounded from the following ingredients given in Table 8.

TABLE 8
The Composition for Tablet Preparation

| Ingredient | Amount |
|---|---|
| Oleandrin-cyclodextrin complex | 6.25 parts |
| Lactose | 79.75 parts |
| Potato starch | 30.00 parts |
| Gelatin | 3.00 parts |
| Magnesium stearate | 1.00 parts |
| Total | 120.0 parts |

PREPARATION: The oleandrin-cyclodextrin complex is intensively milled with ten times its weight of lactose, the milled mixture is admixed with the remaining amount of the lactose and the potato starch, the resulting. mixture is moistened with an aqueous 10% solution of the gelatin, the moist mass is formed through a 1.5 mm-mesh screen, and the resulting granulate is dried at 40 degree C. The dry granulate is again passed through a 1 mm-mesh screen, admixed with the magnesium stearate, and the composition is compressed into 120 mg-tablets in a conventional tablet making machine. Each tablet contains 0.125 mg of oleandrin and is an oral dosage unit composition with effective therapeutic action.

B. Preparation of Coated Pills

The pill core composition is compounded from the ingredients given in Table 9.

TABLE 9
The Composition for Pill Preparation

| Ingredient | Amount |
|---|---|
| Oleandrin-cyclodextrin complex | 6.25 parts |
| Lactose | 26.25 parts |
| Corn starch | 15.00 parts |
| Polyvinylpyrrolidone | 2.00 parts |
| Magnesium stearate | 0.50 parts |
| Total | 50.0 parts |

PREPARATION: The oleandrin-cyclodextrin complex is intensively milled with the lactose, the milled mixture is admixed with the corn starch, the mixture is moistened with an aqueous 15% solution of the polyvinylpyrrolidone, the moist mass is forced through a 1 mm-mesh screen, and the resulting granulate is dried at 40 degree C. and again passed through the screen. The dry granulate is admixed with the magnesium stearte, and the resulting composition is compressed into 50 mg-pill cores which are subsequently coated in conventional manner with a thin shell consisting essentially of a mixture of sugar and talcum and finally polished with beeswax. Each coated pill contains 0.125 mg of oleandrin complexed with hydroxypropyl-cyclodextrin and is an oral dosage unit composition with effective therapeutic action.

C. Preparation of Drop Solution

The solution is compounded from the ingredients given in Table 10.

TABLE 10
The Composition for Drop Solution Preparation

| Ingredient | Amount |
|---|---|
| Oleandrin-cyclodextrin complex | 0.625 parts |
| Saccharin sodium | 0.3 parts |
| Sorbic acid | 0.1 parts |
| Ethanol | 30.0 parts |
| Flavoring | 1.0 parts |
| Distilled water q.s. ad | 100.0 parts |

PREPARATION: The oleandrin-cyclodextrin complex and the flavoring are dissolved in the ethanol, and the sorbic acid and the saccharin sodium are dissolved in the distilled water. The two solutions are uniformly admixed with each other, and the mixed solution is filtered until free from suspended matter. 1 ml of the filtrate contains 0.125 mg of the oleandrin and is an oral dosage unit composition with effective therapeutic action.

D. Preparation of Suppositories

The suppository composition is compounded from the ingredients given in Table 11.

TABLE 11
The Composition for Suppository Preparation

| Ingredient | Amount |
|---|---|
| Oleandrin-cyclodextrin complex | 6.25 parts |
| Lactose | 4.75 parts |
| Suppository base (e.g. cocoa butter) | 1689.0 parts |
| Total | 1700.0 parts |

PREPARATION: The oleandrin-cyclodextrin complex and the lactose are admixed, and the mixture is milled. The milled mixture is uniformly stirred with the aid of an immersion homogenizer into the suppository base, which had previously been melted and cooled to 40 degree C. The resulting composition is cooled at 37 degree C., and 1700 mg-portions thereof are poured into cooled suppository molds and allowed to harden therein. Each suppository contains 0.125 mg of the oleandrin and is rectal dosage unit composition with effective therapeutic action.

E. Preparation of Capsules

The capsule composition is compounded from the following ingredients given in Table 12.

TABLE 12
The Composition for Tablet Preparation

| Ingredient | Amount |
|---|---|
| Oleandrin-cyclodextrin complex | 6.25 parts |
| Lactose | 94.75 parts |
| Micronized Beta-(1,3/16) Glucan | 200.00 parts |
| (Baker's Yeast) | |
| R-Alpha Lipoic Acid | 100.00 parts |
| Total | 400.0 parts |

PREPARATION: The oleandrin-cyclodextrin complex is intensively milled with ten times its weight of lactose, the milled mixture is admixed with the remaining amount of the lactose, the micronized beta-glucan and the R-alpha lipoic acid. The mixed powder is again milled and the composition is filled into 400 mg-capsule in a conventional capsule making machine. Each capsule contains 0.125 mg of oleandrin and is an oral dosage unit composition with effective therapeutic action.

### EXAMPLE 15

### Oral and Suppository Formulations of Proscillaridin-A-Cyclodextrin Complex

100 mg of proscillaridin-A was weighed and placed in a sterile test tube. The proscillaridin-A was dissolved in 2-3 mL of purified absolute ethanol. 50 ml of 9.8% solution of hydroxypropyl-β-cyclodextrin was prepared in a 150 mL sterile beaker and the solution was heated to 70-80 degree centigrade while stirring on a hot plate. The ethanolic solution of proscillaridin-A was slowly added to the beaker with stirring. Within 10-30 minutes, the proscillaridin-A dissolved, leaving a clear solution with no accumulation of crystals. Thus, 100 mg was effectively solubilized in 50 ml of 9.8% solution of hydroxypropyl-β-cyclodextrin. The solution was sterile-filtered through a 0.22 μm filter. The solution was frozen below −40° C. and lyophilized. The lyophilized cake was powdered and used for the tablets, capsule and coated pills formulations and the lyophilized powder is denoted as proscillaridin-A-cyclodextrin complex.

A. Preparation of Tablets

The tablet composition is compounded from the following ingredients given in Table 13.

TABLE 13
The Composition for Tablet Preparation

| Ingredient | Amount |
|---|---|
| Proscillaridin-A-cyclodextrin complex | 12.50 parts |
| Lactose | 73.50 parts |
| Potato starch | 30.00 parts |
| Gelatin | 3.00 parts |
| Magnesium stearate | 1.00 parts |
| Total | 120.0 parts |

PREPARATION: The proscillaridin-A-cyclodextrin complex is intensively milled with five times its weight of lactose, the milled mixture is admixed with the remaining amount of the lactose and the potato starch, the resulting mixture is moistened with an aqueous 10% solution of the gelatin, the moist mass is formed through a 1.5 mm-mesh screen, and the resulting granulate is dried at 40 degree C. The dry granulate is again passed through a 1 mm-mesh screen, admixed with the magnesium stearate, and the composition is compressed into 120 mg-tablets in a conventional tablet making machine. Each tablet contains 0.250 mg of proscillaridin-A and is an oral dosage unit composition with effective therapeutic action.

B. Preparation of Coated Pills

The pill core composition is compounded from the ingredients given in Table 14.

TABLE 14
The Composition for Pill Preparation

| Ingredient | Amount |
|---|---|
| Proscillaridin-A-cyclodextrin complex | 12.50 parts |
| Lactose | 20.00 parts |
| Corn starch | 15.00 parts |
| Polyvinylpyrrolidone | 2.00 parts |
| Magnesium stearate | 0.50 parts |
| Total | 50.0 parts |

PREPARATION: The proscillaridin-A-cyclodextrin complex is intensively milled with the lactose, the milled mixture is admixed with the corn starch, the mixture is moistened with an aqueous 15% solution of the polyvinylpyrrolidone, the moist mass is forced through a 1 mm-mesh screen, and the resulting granulate is dried at 40 degree C. and again passed through the screen. The dry granulate is admixed with the magnesium stearte, and the resulting composition is compressed into 50 mg-pill cores which are subsequently coated in conventional manner with a thin shell consisting essentially of a mixture of sugar and talcum and finally polished with beeswax. Each coated pill contains 0.250 mg of proscillaridin-A complexed with hydroxypropyl-cyclodextrin and is an oral dosage unit composition with effective therapeutic action.

C. Preparation of Drop Solution

The solution is compounded from the ingredients given in Table 15.

TABLE 15
The Composition for Drop Solution Preparation

| Ingredient | Amount |
|---|---|
| Proscillaridin-A-cyclodextrin complex | 1.25 parts |
| Saccharin sodium | 0.3 parts |
| Sorbic acid | 0.1 parts |
| Ethanol | 30.0 parts |
| Flavoring | 1.0 parts |
| Distilled water q.s. ad | 100.0 parts |

PREPARATION: The proscillaridin-A-cyclodextrin complex and the flavoring are dissolved in the ethanol, and the sorbic acid and the saccharin sodium are dissolved in the distilled water. The two solutions are uniformly admixed with each other, and the mixed solution is filtered until free from suspended matter. 1 ml of the filtrate contains 0.250 mg of the proscillaridin-A and is an oral dosage unit composition with effective therapeutic action.

D. Preparation of Suppositories

The suppository composition is compounded from the ingredients given in Table 16.

TABLE 16
The Composition for Suppository Preparation

| Ingredient | Amount |
|---|---|
| Proscillaridin-A-cyclodextrin complex | 12.50 parts |
| Lactose | 4.50 parts |
| Suppository base (e.g. cocoa butter) | 1683.0 parts |
| Total | 1700.0 parts |

PREPARATION: The proscillaridin-A-cyclodextrin complex and the lactose are admixed, and the mixture is milled. The milled mixture is uniformly stirred with the aid of an immersion homogenizer into the suppository base, which had previously been melted and cooled to 40 degree C. The resulting composition is cooled at 37 degree C., and 1700 mg-portions thereof are poured into cooled suppository molds and allowed to harden therein. Each suppository contains 0.250 mg of the proscillaridin-A and is rectal dosage unit composition with effective therapeutic action.

E. Preparation of Capsules

The capsule composition is compounded from the following ingredients given in Table 17.

TABLE 17
The Composition for Tablet Preparation

| Ingredient | Amount |
|---|---|
| Proscillaridin-A-cyclodextrin | 12.50 parts |
| complex | |
| Lactose | 87.50 parts |
| Micronized Beta-(1,3/16) Glucan | 200.00 parts |
| (Baker's Yeast) | |
| R-Alpha Lipoic Acid | 100.00 parts |
| Total | 400.0 parts |

PREPARATION: The proscillaridin-A-cyclodextrin complex is intensively milled with five times its weight of lactose, the milled mixture is admixed with the remaining amount of the lactose, the micronized beta-glucan and the R-alpha lipoic acid. The mixed powder is again milled and the composition is filled into 400 mg-capsule in a conventional capsule making machine. Each capsule contains 0.250 mg of proscillaridin-A and is an oral dosage unit composition with effective therapeutic action.

Likewise, the amount of active ingredient in these illustrative examples may be varied to achieve the dosage unit range set forth above, and the amounts and nature of the inert pharmaceutical carrier ingredients may be varied to meet particular requirements.

While the present invention has been illustrated with the aid of certain specific embodiments thereof, it will be readily apparent to others skilled in the art that the invention is not limited to these particular embodiments, and that various changes and modifications may be made without departing from the spirit of the invention or the scope of the appended claims.

### REFERENCES

Müller B W, Waaler T, Helm H and Anderson A: Complex formation of β- and g-cyclodextrins with digitoxin, Acta Pharm. Nord.1992; 4: 313-317.

Albers E and Müller B W: Cyclodextrin derivatives in pharmaceutics, Crit Rev Ther Drug Carrier Syst. 1995; 12:311-37.

Uekama K, Fujinaga T, Hirayama F, Otagiri M, Yamasaki M, Seo H, Hashimoto T, Tsuruoka M: Improvement of the oral bioavailability of digitalis glycosides by cyclodextrin complexation, J Pharm Sci 1983; 72: 1338-41.

Okada Y, Koizumi K: Some properties and the inclusion behavior of three positional isomers of 6(1),6n-di-O-alpha-D-glucosyl-cyclomaltoheptaoses (beta-cyclodextrins), Chem Pharm Bull (Tokyo) 1998; 46: 319-23.

Ueda H, Wakamiya A, Endo T, Nagase H, Tomono K, Nagai T: Interaction of cyclomaltononaose (delta-CD) with several drugs, Drug Dev Ind Pharm 1999; 25: 951-4.

Bekers et al.: “Stabilization of Mitomycins on Complexation With Cyclodextrins in Aqueous Acidic Media”, Intl. J. of Pharmaceutics, 52, 239 (1989).

Abe F and Yamauchi T: Phytochemistry, 31, 2459 (1992).

Allen T M, Hansen C, Martin F J, Redemann C and Yau-Young A: Liposome containing a synthetic lipid derivative of poly(ethylene glycol) show prolonged circulation half-lives in vivo. Biochim. Biophys. Acta, 1066, 29-36 (1991).

Baba et al.: Proc. Nat. Acad. Sci., USA, 85, 6132 (1988).

Baba et al.: Antiviral Res., 9, 335 (1988).

Blum L M and Rieders F: Oleandrin distribution in a fatality from rectal and oral Nerium oleander extract administration. J Anal Toxicol, 1987, 11(5): 219-221.

Chopra R N, Nayar S L and Chopra I C: Glossary of Indian Medicinal Plants, CSIR, New Delhi (1956), page 175.

Dey K L and Bahadur R: Indigenous Drugs of India, International Book Publishers, India (1984).

Dobereiner H G, Kas J, Noppl D, Sprenger I & Sackmann E: Biophys. J 65, 1396-140 (1993).

Drummond D C et al: Optimizing Liposomes for Delivery of Chemotherapeutic Agents to Solid Tumors. Pharmacological Reviews 51, 691-743, 1999 and the references cited therein.

Dymock W: Pharmacographia Indica, 2, 398, (1890).

Font-Quer P: (1979) Plantas medicinales. Barcelona, Ed. Labor SA.

Gabizon A. and Papahadjopoulos D: Liposome formulations with prolonged circulation time in blood and enhanced uptake by tumors. Proc. Natl. Acad. Sci. (Wash.) 85, 6949-53 (1988).

Gordon K B, Tajuddin A, Guitart J, Kuzel, T M, Eramo L R, and Von Roenn J: Cancer, 75, 2169-73 (1995).

Hanada R, Abe F and Yamauchi T: Phytochemistry, 31, 2459 (1992).

Hanada R, Abe F and Yamauchi T: Phytochemistry, 31, 3183 (1992).

Hardin J W and Arena J M: (1974) Human poisoning from native a cultivated plants, 2nd ed. Kingsport, Tenn., Duke University Press.

Huxley A: ed-in-chief (1992) The New Horticultural Society dictionary of gardening, vol. 3. London, MacMillan.

Julliano R L and Stamp D: Pharmacokinetics of liposome-encapsulated anti-tumor drugs. Biochem. Pharm. 27, 21-27 (1978).

Kingsbury J M: (1964) Poisonous plants of the United States and Canada. Englewood Cliffs, N.J. Prentice Hall.

Kirtikar K R and Basu B D: Indian Medicinal Plants, Internatiional Book Ditributors, India (1987).

Krasso et al: Helv. Chim. Acta, 46, 1691 (1963).

Lasic D D, & Martin F: eds. (1995) Stealth Liposomes, CRC, Boca Raton, Fla.

Lasic D D and Paphadjopoulos D: Medical Applications of Liposomes, Amsterdam, Elsevier, 1998.

Lasic D D: Liposomes: From Physics to Applications. Amsterdam, Elsvier, 1993.

Lee et al: “Serum Albumin Beads: An Injectable, Biodegradable System for the Sustained Release of Drugs” Science, 213:233-235 (1981).

Leucuta et al: “Albumin microspheres as a drug delivery system for epirubicin: pharmaceutical, pharmacokinetic and biological aspects” International Journal of Pharmaceutics, 41:213-217 (1988).

Madden T D, Harrigan P R, Tai LCL, Bally M B, Mayer L D, Redelmeier T E, Loughrey H C, Tilcock CPS, Reinish L W and Cullis P R: The accumulation of drugs within large unilamellar vesicles exhibiting a proton gradient: a survey, Chem. Phys. Lipids, 53, 37-46 (1990).

Manna S K, Sah N K, Newman R A, Cisneros A, Aggarwal B B: Cancer Res, 60(14): 3838-47, 2000

McConkey D J, Lin Y, Nutt L K, Ozel H Z, Newman R A: Cancer Res, 60(14): 3807-12, 2000

Olson F, Hunt C, Szoka FC: Preparation of liposomes of defined size distribution by extrusion through polycarbonate membrane. BioChim. Biophys. Acta, 557, 9-23 (1979).

Olson F, Mayhew E, Maslow, Rustum Y and Szoka F: Characterization, toxicity and therapeutic efficacy of adriamycin entrapped in liposomes. Europ. J. Cancer. Clin. Oncol., 18, 167-76 (1982).

Pathak S, Multani A S, Narayan S, Kumar V, Newman R A: Anticancer Drugs, 11(6): 455-63, 2000

Pearn J: (1987) Oleander poisoning. In: Covacevich J, Davie P and Pearn J: eds (1987) Toxic plants & animals; a guide for Australia. Brisbane, Queensland Museum, pp 37-49.

Rahman A., Kessler A., More N, Skies B, Rowden G, Wooley P and Schein P S: Liposomal protection of adriamycin induced cardiotoxicity in mice. Cancer Res., 40, 1532-37 (1982).

Reynolds J E F: ed (1989) Martindale: the extra pharmacopoeia, 29th ed. London, The Pharmaceutical Press.

Schvartsman S: (1979) Plantas venenosas. Sarvier Sao Paulo.

Sharma A, Sharma U S, and Straubinger R M: Paclitaxel-liposomes for intracavitary therapy of intraperitoneal P388 leukemia., Cancer Lett 107: 265-272 (1996).

Sharma A, Mayhew E, Bolcsak L, Cavanaugh C and Harmon P: Activity of paclitaxel liposome formulations against human ovarian tumor xenografts. Int J Cancer 71: 103-107(1997).

Shaw D and Pearn J: (1979) Oleander poisoning. Med J Aust, 2: 267-269.

Siddiqui B S, Begum S, Hafeez F and Siddiqui S: Phytochemistry, 28, 1187 (1995).

Siddiqui B S, Begum S, Siddiqui S and Lichter W: Phytochemistry, 39, 171 (1995).

Siddiqui S, Begum S, Hafeez F and Siddiqui B S: Phytochemistry, 28, 1187 (1989).

Siddiqui S, Begum S, Siddiqui B S and Hafeez F: J. Nat. Products, 52, 229 (1988).

Siddiqui S, Hafeez F, Begum S and Siddiqui B S: J. Nat. Products, 49, 1086 (1987).

Siddiqui S, Hafeez F, Begum S and Siddiqui B S: J. Nat. Products, 51, 229 (1988).

Siddiqui S, Hafeez F, Begum S, and Siddiqui B S: Phytochemistry, 26, 237 (1987).

Siddiqui S, Siddiqui B S, Begum S, and Hafeez F: Plant Med., 55, 292 (1989).

Siddiqui S, Siddiqui B S, Hafeez F and Begum S: Planta Med., 53, 424 (1987).

Siddiqui S, Siddiqui B S, Hafeez F. and Begum S: Planta Med., 54, 232 (1988).

Smith J A, Madden T, Vijjeswarapu M, Newman R A: Biochem Pharmacol, 62(4): 469-72, 2001

Smith J A, Madden T, Vijjeswarapu M, Newman R A: Biochem Pharmacol, 62(4): 469-72, 2001

Suffness, M: (Ed.) Taxol, Science and Applications, Chapter 9, CRC (1995).

Taylor A., McKenna G F and Burlage H M: Texas Reports on Biology and Medicine, 14, 538 (1956).

Wang X, Plomley J B, Newman R A, Cisneros A: Anal Chem, 72(15): 3547-52, 2000

William S S, Alosco T R, Mayhew E, Lasic D D, Martin F J and Bankert R B: Arrest of human lung tumor xenograft growth in severe combined immunodeficient mice using doxorubicin encapsulated in sterically stabilized liposomes. Cancer Res., 53, 3964-67 (1993).

Woodle M C, Allen T M, Mayhew E, and Uster P S: In vivo efficacy of vincristine entrapped in long-circulating liposomes, Proc. Am. Assoc. Cancer Res., 33, 446 (1992).

Yeh J Y, Huang W J, Kan S F, Wang P S., Inhibitory effects of digitalis on the proliferation of androgen dependent and independent prostate cancer cells, J. Urol., 2001 166(5): 1937-42.

D. Stewart, A. Mobasheri and M. B. A. Djamgoz, Immuno-characterisation of Na⁺,K⁺-ATPase α-subunit(s) in rat prostate cancer cell lines, The Journal of Physiology (2000), 525P, pp. 96P.

Grimes J A, Fraser S P, Stephens G J, Downing J E, Laniado M E, Foster C S, Abel P D, Djamgoz M B., Differential expression of voltage-activated Na⁺ currents in two prostatic tumour cell lines: contribution to invasiveness in vitro., FEBS Lett 1995; 369:290-4.

Repke K R H, Schönfeld W, Weiland J, Megges R, Hache, A: In Design of Enzyme Inhibitors as Drugs; Sandler, M.; Smith, H. J., Eds.; Oxford University Press: Oxford, 1989; pp 435-502.

Thomas R E, Gray P, Andrews J: In Advances in Drug Research; Testa, L. B., Ed.; Academic Press: London, 1990; Vol. 19, pp 313-575.

Blaustein M P: Rev. Physiol. Biochem. Pharmacol. 1974, 70, 33-82.

Skou J C: Biochim. Biophys. Acta 1957, 23, 394-401.

Albers R W: Annu. Rev. Biochem. 1967, 36, 727-756.

Post R L, Kume S, Tobin T, Orcutt T, Sen A K: J. Gen. Physiol. 1969, 54, 306S-326S.

Geering K: Curr. Op. Nephrol. Hypertens. 1997, 6, 434-439.

Thomas R E: In Molecular Structure and Biological Activity of Steroids; Bohl M, Daux W L, Eds.; CRC Press: Boca Raton, 1992; pp 399-464.

Lee C O: Am. J. Physiol. 1985, 249, 367-378.

Gillis R A, Quest J A: In Cardiac Glycosides; Erdmann E, Greeff K, Skou J C, Eds.; Steinkopff: Darmstadt, 1986; pp 347-356.

Concepción P. Melero, Manuel M and Arturo S F: A Short Review on Cardiotonic Steroids and Their Aminoguanidine Analogues. Molecules 2000, 5, 51-81

Wald H, Scherzer P, Popovtzer M M: Enhanced renal tubular ouabain-sensitive ATPase in streptozotocin diabetes mellitus, Am J Physiol 1986; 251: F164-70

Jennings P E, Wilkins M R, West M J, Kendall M J, Barnett A H: Sodium transport across erythrocyte membranes in diabetes mellitus. Sodium transport across erythrocyte membranes in diabetes mellitus, Diabetes Res 1986; 3:407-10

Kjeldsen K, Braendgaard H, Sidenius P, Larsen J S, Norgaard A: Diabetes decreases Na⁺-K⁺ pump concentration in skeletal muscles, heart ventricular muscle, and peripheral nerves of rat, Diabetes 1987; 36:842-8

Baldini P, Incerpi S, Lambert-Gardini S, Spinedi A, Luly P: Membrane lipid alterations and Na⁺-pumping activity in erythrocytes from IDDM and NIDDM subjects, Diabetes 1989; 38:825-31

Kuriyama S, Tokudome G, Tomonari H, Utsunomiya Y, Matsui K, Hashimoto T, Sakai O: Differential regulation of cation transport of vascular smooth muscle cells in a high glucose concentration milieu, Diabetes Res Clin Pract 1994; 24:77-84

Raccah D, Dadoun F, Coste T, Vague P: Decreased Na/K ATPase ouabain binding sites in red blood cells of patients with insulin-dependent diabetes and healthy north African control subjects: relationship with diabetic neuropathy, Horm Metab Res 1996; 28:128-32

Zolese G, Rabini R A, Fumelli P, Staffolani R, Curatola A, Kvasnicka P, Kotyk A, Cester N, Mazzanti L: Modifications induced by insulin-dependent diabetes mellitus on human placental Na⁺/K⁺-adenosine triphosphatase, J Lab Clin Med. 1997; 130:352-3.

Gerbi A, Maixent J M, Barbey O, Jamme I, Pierlovisi M, Coste T, Pieroni G, Nouvelot A, Vague P, Raccah D: Alterations of Na,K-ATPase isoenzymes in the rat diabetic neuropathy: protective effect of dietary supplementation with n-3 fatty acids, J Neurochem 1998;71:732-40

Wild G E, Thompson J A, Searles L, Turner R, Hasan J, Thomson A B: Small intestinal Na⁺, K⁺-adenosine triphosphatase activity and gene expression in experimental diabetes mellitus, Dig Dis Sci 1999;44:407-14

Takigawa T, Yasuda H, Terada M, Maeda K, Haneda M, Kashiwagi A, Kitasato H, Kikkawa R: Increases in K⁺ conductance and Ca²⁺ influx under high glucose with suppressed Na⁺/K⁺-pump activity in rat myelinated nerve fibers, Neuroreport 2000; 11:2547-51

Sennoune S, Gerbi A, Duran M J, Grillasca J P, Compe E, Pierre S, Planells R, Bourdeaux M, Vague P, Pieroni G, Maixent JM: Effect of streptozotocin-induced diabetes on rat liver Na⁺/K⁺-ATPase, Eur J Biochem 2000; 267:2071-8.

Shiratori O: Growth inhibitory effect of cardiac glycosides and agly cones on neoplastic cells: in vitro and in vivo studies. Gann 1967, 58:521-528

Repke K R H: The role of the Na⁺/K⁺ pump in normal and cancer cell proliferation. In: Biomemebranes, Basic and Medical Research (Edited by Benga G, Tager J M) Berlin, Springer Verlag 1988, 161-176

Repke K R, Schon R, Megges R, Wetland J, Nissen E, Matthes E: Potential suitability of Na⁺/K⁺-transporting ATPase in pre-screens for anti-cancer agents. Anticancer Drug Des 1995, 10:177-87

Cassady J M, Suffness M M: Terpenoid antitumor targets. In: Anti cancer agents based on natural product models (Edited by Cassady J M, Douros J D) New York, Academic Press 1980, 201-269

Haux J: Digitoxin is a potential anticancer agent for several types of cancer. Med Hypotheses 1999, 53:543-548

Haux J, Lam M, Marthinsen A B L, Strickert T, Lundgren S: Digitoxin, in non toxic concentrations, induces apoptotic cell death in Jurkat T cells in vitro. Z Onkol 1999, 31:14-20

Haux J, Solheim O, Isaksen T, Angelsen A: Digitoxin, in non-toxic concentrations, inhibits proliferation and induces cell death in prostate cancer cell lines. Z Onkol 2000, 32:11-16

Kawazoe N, Aiuchi T, Masuda Y, Nakajo S, Nakaya K: Induction of apoptosis by bufalin in human tumor cells is associated with a change of intracellular concentration of Na⁺ions. J Biochem (Tokyo) 1999, 126:278-286

Johansson S, Lindholm P, Gullbo J, Larsson R, Bohlin L, Claeson P: Cytotoxicity of digitoxin and related cardiac glycosides in human tumor cells. Anticancer Drugs 2001, 12:475-483

Raloff J: Jamming Prostate Cancer's Transmission. Science News 1995, 147, 134.

Pienta et al.: Inhibition of Spontaneous Rat Prostate Cancer By Oral Administration of Modified Citrus Pectin. J. Nat'l Cancer Institute, 1995, 87, 348-53.

Inohara et al.: Effects of Natural Complex Carbohydrate (Citrus Pectin) On Murine Melanoma Cell Properties Related to Galectin-3 Functions. Glycocon J, 1994, 11, 527-532.

Platt, D, and Raz, A: Modulation of the lung colonization of B16-F1 melanoma cells by citrus pectin. J. Natl Cancer Inst, 1992, 84, 438-42.

Masaru I, Tsutomu W, and Tadataka N: Bufadienolides and a New Lignan from the Bulbs of Urginea maritima. Chem. Pharm. Bull. 2001, 49, 282-286.

Kopp B, Krenn L, Draxler M, Hoyer A, Terkola R, Vallaster P, Robien W: Phytochemistry, 1996, 42, 513-522.

Mitsuhashi H, Tanaka O, Nozoe S, Nagai M. (ed.): Chemistry of Organic Natural Products. 4th ed, Nankoudou Press, Tokyo, 1994, 168-174.

Schoenfeld W, Weiland J, Lindig C, Masnyk M, Kabat M M, Kurek A, Wicha J, Repke K R H: Naunyn Schmiedeberg's Arch. Pharmacol., 1985, 329, 414-426.

Majinda R R T, Waigh R D, Waterman P G: Planta Med., 1997, 63, 188-190.

Kopp B, Unterluggauer M, Robien W, Kubelka W: Planta Med., 1990, 56, 193-197.

Krenn L, Kopp B, Deim A, Robien W, Kubelka W, Planta Med., 1994, 60, 63-69.

Krishna Rao R V, Rangaswami S: Tetrahedron Lett., 1967, 4563-4565.

Krenn L, Jambrits M, Kopp B: Planta Med., 1988, 54, 227-232.

Tanase T, Nagatsu A, Murakami N, Nagai S, Ueda T, Sakakibara J, Ando H, Hotta Y, Takeya ., Asano M: Chem. Pharm. Bull., 1994, 42, 2256-2262.

Verbiscar A J, Patel J, Banigan T F, Schatz R A J: Agric. Food Chem., 1986, 34, 973-979.

Shimada K, Umezawa E, Nambara T, Kupchan SM: Chem. Pharm. Bull., 1979, 27, 3111-3114.

Jha S, Sen S: Phytochemistry, 1981, 20, 524-526.

Lichti H, Niklaus P, von Wartburg A: Helv. Chim. Acta, 1973, 56, 2083-2087.

1. A composition comprising at least one digitalis glycoside and a cyclodextrin.

2. The composition of claim 1, further defined as a pharmaceutical composition comprising the at least one digitalis glycoside and an amorphous cyclodextrin.

3. The composition of claim 2, wherein the pharmaceutical composition comprises one or more excipients.

4. The composition of claim 2, wherein pharmaceutical composition comprises one or more pharmaceutically acceptable antioxidants.

5. The composition of claim 2, wherein the pharmaceutical composition comprises one or more pharmaceutically acceptable preservatives.

6. The composition of claim 2, wherein the pharmaceutical composition comprises one or more pharmaceutically acceptable buffering agents.

7. The composition of claim 2, wherein the pharmaceutical composition comprises one or more pharmaceutically acceptable polysaccharides.

8. The composition of claim 3, wherein the said excipients comprises mannitol, sorbitol, fructose, glucose, lactose, sucrose, trehalose or any other water soluble sugar.

9. The composition of claim 4, wherein the said antioxidants comprise ascorbic acid, sodium ascorbate, sodium bisulfate, sodium metabisulfate, curcumin, curcumin derivatives, ursolic acid, resveratrol, resveratrol derivatives, alpha-lipoic acid or monothio glycerol.

10. The composition of claim 5, wherein the said preservatives comprise a methylparaben, methylparaben sodium, propylparaben, propylparaben sodium, benzalkonium chloride, or benzthonium chloride.

11. The composition of claim 6, wherein the said buffering agents comprise monobasic and dibasic sodium phosphate, sodium benzoate, potassium benzoate, sodium citrate, sodium acetate or sodium tartrate.

12. The composition of claim 7, wherein the polysaccharides comprise dextran sulfate, pectin, modified pectin, insoluble 1,3-β-D glucan, micronized 1,3-β-D glucan, soluble 1,3-β-D glucan, phosphorylated 1,3-β-D glucan, aminated 1,3-β-D glucan or carboxymethylated 1,3-β-D glucan, sulfated 1,3-β-D glucan.

13. The composition of claim 1, wherein the digitalis glycoside is oleandrin, neriifolin, odoroside A or H, ouabain (G-strophantin), cymarin, sarmentocymarin, periplocymarin, K-strophantin, thevetin A, cerberin, peruvoside, thevetosin, thevetin B, tanghinin, deacetyltanghinin, echujin, hongheloside G, honghelin, periplocin, strophantidol, nigrescin, uzarin, calotropin, cheiroside A, cheirotoxin, euonoside, euobioside, euomonoside, lancetoxin A and B, kalanchoside, bryotoxin A-C, bryophyllin B, cotiledoside, tyledoside A-D, F and G, orbicuside A-C, alloglaucotoxin, corotoxin, coroglaucin, glaucorin, scillarene A and B, scilliroside, scilliacinoside, scilliglaucoside, scilliglaucosidin, scillirosidin, scillirubrosidin, scillirubroside, proscillaridin A, methyl-proscillaridin A, rubelin, convalloside, convallatoxin, bovoside A, glucobovoside A, bovoruboside, antiarin A, helleborin, hellebrin, adonidin, adonin, adonitoxin, thesiuside, digitoxin, gitoxin, gitalin, digoxin, F-gitonin, digitonin, lanatoside A-C, bufotalin, bufotalinin, bufotalidin, pseudobufotalin, acetyl-digitoxin, acetyl-oleandrin, beta-methyldigoxin or alpha-methyldigoxin.

14. The composition of claim 13 wherein the digitalis glycoside is oleandrin.

15. The composition of claim 13 wherein the digitalis glycoside is odoroside A or odoroside H.

16. The composition of claim 13 wherein the digitalis glycoside is digitoxin.

17. The composition of claim 13 wherein the digitalis glycoside is proscillaridin A.

18. The composition of claim 13 wherein the digitalis glycoside is methyl-proscillaridin A.

19. The composition of claim 13 wherein the digitalis glycoside is neriifolin.

20. The composition of claim 2 wherein said amorphous cyclodextrin has a degree of substitution of 2 to 7.

21. The composition of claim 1 wherein the ratio by weight of digitalis glycoside to amorphous cyclodextrin is 0.01 to 1.

22. A process for preparing a pharmaceutical composition comprising admixing at least one digitalis glycoside with a cyclodextrin and rendering said composition pharmaceutically acceptable.

23. The process of claim 22, wherein the composition is rendered sterile by filtration.

24. The process of claim 22, wherein the composition is freeze-dried or lyophilized.

25. A method of treating a cell proliferative disease in a subject comprising administering an amount of the composition of claim 1 that is effective to treat the cell proliferative disease.

26. The method of claim 25, wherein the subject is a human subject.

27. The method of claim 25, wherein the composition comprises the digitalis glycoside at a concentration of from 0.01 mg per mL to 10 mg per mL.

28. The method of claim 27, wherein the digitalis glycoside is at a concentration of from 0.04 mg per mL to 5 mg per mL.

29. The method of claim 25 wherein the composition is administered to the subject intramuscularly, intravenously or subcutaneously.

30. The method of claim 25, wherein the composition is administered orally, intranasally, rectally or vaginally.
